(19) United States
(12) Patent Application Publication
(10) Pub. No.: US 2024/0159764 A1
(43) Pub. Date: May 16, 2024

(54) COMPOSITIONS AND METHODS FOR SELECTIVE CAPTURE, PURIFICATION, RELEASE AND ISOLATION OF SINGLE CELLS

(71) Applicant: OneCell Diagnostics, Inc., Cupertino, CA (US)

(72) Inventors: Jayant Jagannath Khandare, PUNE (IN); Ganesh Khutale, PUNE (IN); Atul Bharde, PUNE (IN); Snigdha Nadagouda, PUNE (IN); Gourishankar Rajshekhar Aland, PUNE (IN); Gowhar Shafi, Mumbai (IN)

(21) Appl. No.: 18/165,425

(22) Filed: Feb. 7, 2023

(30) Foreign Application Priority Data
Nov. 12, 2022 (IN) 202241064961

(51) Int. Cl. G01N 33/574; C12N 5/09; G01N 33/552

(52) U.S. Cl. CPC G01N 33/57492; C12N 5/0694; G01N 33/552; G01N 2333/70589; G01N 2333/70596

(57) ABSTRACT

This disclosure relates to adsorbent compositions and methods for selective capture, purification, and release of marker cells for cancer, more particularly, circulating rare cells exemplified by circulating tumour cells followed by their isolation as single cells. These adsorbent compositions include functionalized substrates linked to the ligands which bind non-covalently to a marker cell for cancer, through at least one linkage cleaved at controlled rate in the presence of specific stimuli, and also linked to ligands which bind non-covalently to leukocytes through non-cleavable linkages. The capture of marker cell for cancer and release may be monitored using microscopy. Methods for the capture and controlled release of these marker cells using these adsorbent compositions lead to isolation of single cells useful in diagnosis, prognosis and in the screening of therapeutic treatment of diseases, especially oncological diseases, genomics, single cell whole genome amplification, multi-omics, transcriptomics, proteomics, to identify genetic, genomic signatures, mutations.

### CROSS-REFERENCE TO RELATED APPLICATIONS

This application claims priority to and the benefit of Indian Patent Application No. 202241064961, filed 12 Nov. 2022 and titled COMPOSITIONS AND METHODS FOR SELECTIVE CAPTURE, PURIFICATION, RELEASE AND ISOLATION OF SINGLE CELLS, which incorporated by reference herein in its entirety.

### BACKGROUND

### Field of the Invention

This disclosure relates to adsorbent compositions useful for selective capture, purification, as well as release of marker cells for cancer such as circulating rare cells exemplified by circulating tumour cells (CTCs) followed by their isolation as single cells. The adsorbent compositions and methods disclosed herein are useful for isolating single cells which are useful in evaluating the status of the patient, monitoring response to the treatment and assessing outcome of the treatment by analysing the features of the isolated single cells from the patient's blood. The isolated single cells are useful in selecting methods for personalized treatment of diseases including oncological diseases.

### Background Information

Many systemic diseases such as cancers and neurological disorders are characterized by high cellular and genomic alterations and complexities (Frew, I J. et al. Ann Rev Pathol. 2015; 10, 263; Young, A L. et al. Nat Commun. 2018; 9, 4273). Cancer is a difficult disease to treat and manage clinically. This is because tumor biology is continuously evolving due to the genomic instability of tumor cells. In addition, cytotoxic therapy imparts a selection pressure that modifies the biological growth pathways of tumor cells and leads to dynamic alterations of therapeutic targets (Venkatesan, S. et al. Cold Spring Harb Perspect Med. 2017; 7). This eventually results in developing cancer resistance to the administered therapy (Foo, et. al. J Theor Biol. 2014; 355, 10-20).

Another characteristic aspect that exacerbates the clinical complexity of cancer is tumor heterogeneity (Almendro V et al. Ann Rev Pathol. 2013; 8, 277-308). Investigating heterogeneity in tumor cells is useful to determine the clonal population, metastatic behaviour and occurrence of the cancer stem cells. This helps understand the significant variation in treatment response among patients suffering from the same type of cancer. In the absence of such an understanding, some patients may benefit while others show severe side effects without significant clinical benefits in response to the same cancer treatment. Due to heterogeneity, different tumor cells residing in the same tumor show varying responses to the administered therapy (Sun et al. Acta PharmacologicaSinica. 2015; 36; 1219-1227). The aforesaid traits of cancer that are responsible for dismal clinical outcomes can be better understood by investigating cancer cells at the single-cell level and subsequently designing personalized treatments.

Technological advancements in multi-omics platforms, including next-generation sequencing (NGS), help identify how tumor-driving gene alterations dysregulate protein-protein interactions within human cells harbouring mutations. However, the data output from these platforms is significantly biased towards the population-based input (Manuel, L. et al. Per Med. 2014;11; 523). This problem can be greatly minimized by isolating individual circulating tumor cells (CTCs) and performing multi-omics analysis thereon.

CTCs serve as a privileged gateway to study mechanisms of metastasis and have prognostic and predictive value in breast, and other cancers. (Hwang, W L. et al. Adv Drug Deliv Rev. 2018; 125, 122-131). Therefore, analysis of single and pooled CTCs is a powerful tool in clinical diagnosis and prognosis and has the potential for developing CTC-targeting therapies. These single and pooled CTC analyses at highest precision genomic profiling allow the investigation of tumor heterogeneity, driver mutations, microsatellite instability, alterations, fusions, loss of heterozygosity etc. providing insights into tumor evolution and treatment resistance (Francoise Rothé, et al. NPJ Breast Cancer. 2022; 8(1), 79).

CTCs are unique resources for the real-time monitoring of active tumor biology in cancer research. Moreover, CTCs are biomarkers with a biological function in metastatic development, and their analyses may highlight mechanisms of cancer progression (Catherine Alix-Panabieres, et al. J Mol Med (Berl). 2017; 95(2), 133-142). Also, CTCs have great utility in therapeutic selection, monitoring of cancer patients, and fundamental understanding of metastasis and cellular heterogeneity of CTCs (Yu-Heng Cheng, et al. Nat Commun. 2019; (10), 2163).

Thus, CTCs represent unique spatial and temporal heterogeneity, genomic profile and plasticity as the primary tumor. Therefore, isolating CTCs from blood offers a viable and practical alternative to performing multi-omics analysis on individual cancer cells. However, isolating and enumerating the CTCs at single cell level without any interventions like cell fixing, is highly challenging.

Detection and molecular characterization of CTCs is important for the stratification of cancer patients for prognostic benefits (Lin, et al. Sig Transduct Target Ther. 2021; 6, 404). A variety of methods have been reported for the isolation of CTCs from peripheral blood of cancer patients. This includes immunomagnetic CTC separation methods such as CellSearch platform for prognosis of breast, colorectal, and prostate cancer (Miller, et al. J. Oncol. 2010, 617421; U. S. Pat. Appl. 7901950; U. S. Pat. Appl. 8337755; U. S. Pat. Appl. 2013/0157347), and the OncoDiscover technology (PCT/IB2016/050779). Other methods to isolate CTCs based on their physical properties such as size and shape using filtration, sedimentation, and fluidics-based methods such as microfluidic devices and flow cytometry, have been developed. To obtain a personalized profile of cancer, isolating and collecting CTCs from cancer patients at a single-cell level is highly desirable. This will be helpful to create an individualized CTC bank for generating patient-specific tumor genome landscape and transcriptional analysis via multi-omics analysis (Negishi, R et al. Commun Biol.; 2022, 5, 20).

A variety of methods have been devised to obtain rare cells from heterogenous biological samples, for example, CTCs, from peripheral blood of cancer patients. Single-cell analysis can be performed by isolating a whole cell or cell-specific nuclei or organelles. A number of approaches have been described for sorting single cells. These include laser capture microdissection (LMD) (Espina, V. et al. Expert Rev Mol Diagn. 2007; 7, 647), fluorescence-activated cell sorting (Gross, A. et al. Int J Mol Sci. 2015; 16, 16897), magnetic isolation (Citri, A. et al. Nat Protoc. 2012, 7, 111), microfluidics (Lecault, V. et al. CurrOpin Chem Biol. 2012; 16, 381) immunopanning and limiting dilution method (Hu, et al. Front Cell Dev Biol. 2016, 4, 116).

The emergence of enrichment and single-cell isolation technologies have allowed for downstream analysis of CTC with greater depth of characterisation, which provides crucial information of the primary tumour. Thus, use of single-cell analysis technologies can enhance the analysis of CTCs and may identify the potential clinical use of CTCs as a cancer biomarker. The technologies for isolation and analysis of single cell CTCs depends on cell-loss, study cost per CTC, and workflow complexity (Payar Radfar, et al. Trends in Biotechnology, 2022; 40(9), 1041-1060). Present technologies suffer from huge analysis costs and cell-loss during handling of the low number of CTCs. Additionally, different bio-physical features of CTC clusters often lead to difficulties in analysing them.

Microfluidic technologies have been developed, which define the specific interaction between the surface of the substrate and CTCs (Asghari, et al. BiotechnolBioproc E. 2020; 26, 529). Electric fields have also been applied to dissociate tissues into viable cells (E. Celeste, Welch. et al. Scientific reports; 2022, 12, 10728).

Yu-Heng Cheng et. al. developed Hydro-Seq, a platform technology, which enables single-cell transcriptome analysis of rare and limited cell populations such as CTCs. This technology provides downstream analysis capability for molecular characterization of CTCs at single-cell resolution beyond CTC enrichment (Yu-Heng Cheng, et al. Nat Commun. 2019; (10), 2163).

Also, Gao, et al. describes a cycling enzymatic processing method aiming to harvest qualified single-cell suspensions for single-cell RNA-sequencing (scRNA-seq) (Manman Gao, et al. BMC Molecular and Cell Biology; 2022, 23, 32). This method resulted in a single cell suspension from a cartilage tissue.

U.S. patent application Ser. No. 14/890,918 discloses a microfluidic chip utilizing nanoparticle-based multivalent binding for the capture and release of circulating tumor cells (CTCs) from the blood of a pancreatic cancer patient. This method yielded CTCs at the single cell level with high purity but relatively slower rate.

U.S. patent application Ser. No. 13/284,482 discloses a method and an automated robotic device for the capture and separation of circulating tumor cells (CTCs) or other biological structures in blood samples utilizing a two-step process that involves capturing CTCs with antibody conjugated super magnetic beads and subsequently releasing it into the capture solution. The said method resulted in a single CTC release but required an additional purification step.

U.S. patent application Ser. No. 16/832,396 discloses a novel microfluidic chip-based method utilizing a selective surface coating to capture and release a biological substance (e.g., cancer cells, fetal cells, bacteria, viruses, etc.) as well as the removal of non-specific cells or protein by releasing the same from the surface of the microstructures. Although a versatile method to isolate single biological cell, this method required uniform surface coating to minimize non-specific cell binding that contaminate the purity of isolated single cell.

PCT/US2012/022248 discloses a device utilizing cell picking techniques for capture, isolation of single circulating tumor cell (CTC), and application of single-cell omics from peripheral blood of a lung cancer patient.

U.S. patent application Ser. No. 12/223,351 discloses methods for detecting circulating tumor cells in a mammalian subject. The methods of detection or diagnosis indicate the presence of metastatic cancer or early-stage cancer.

PCT/US2010/003431 discloses computational approaches utilizing data from non-rare cells to detect rare cells such as circulating tumor cells (CTCs). The invention is applicable at two distinct stages of CTC detection; the first being to make decisions about data collection parameters and the second being to make decisions during data reduction and analysis.

Park et al. (J. Am. Chem. Soc. 2017; 7, 2741) disclose a microfluidic chip utilizing a selective chemical ligand-exchange reaction for the capture and release of circulating tumor cells (CTCs) from the blood of a cancer patient. This was achieved by engineering a chemical ligand-exchange reaction to release cells attached to a gold nanoparticle coating bound to the surface of a herringbone microfluidic chip.

Wang et al. (Adv. Mater. Interfaces. 2021; 8, 2101191) disclose a method comprising degradable magnetic nanocomposite for charge-based CTC capture and pH-controlled release of CTC from the spiked sample and clinical peripheral blood sample. Here, the polyelectrolyte functionalized CaCO₃ coated magnetic nanocrystal clusters are developed as the nanoplatform for multi-subpopulations isolation and pH-dependent release of CTCs.

Cheng et al. (Anal Chem. 2017; 89, 7924) disclose a method for the capture and release of individual and clusters of CTCs using a thermo-responsive three-dimensional (3D) scaffold chip. This allows global DNA and RNA methylation analysis of collected single CTC and CTC clusters.

The afore-mentioned techniques, however, are limited in their ability to isolate viable single CTCs and other rare cells from blood. The methods of isolation of single marker cells for cancer may result in contamination with other non-targeted cells, e.g., leukocytes, and the contaminant will overshadow the molecular signatures specific to marker cells for cancer, such as CTCs. The detection of isolated CTCs according to prior methods is also not possible without fixing them. However, such cells cannot be used for further downstream genomic processing. As such, there continues to be a need in the art for a method for selective capture, release, and collection of individual intact CTCs for downstream physical and biological characterization and determining genetic and phenotypic changes occurring in tumor for clinical benefit of cancer patients.

### SUMMARY

The present disclosure provides compositions useful to isolate marker cells for cancer at the single-cell level from heterogeneous biological and clinical blood samples. Also disclosed are methods to capture and release intact circulating tumour cells (CTCs) and isolate the same as single cells from the blood samples of cancer patients. The single cells thus isolated are useful, for example, for downstream multi-omics applications including identifying and determining genomic mutations in patients of various epithelial cancers, for example, colon, rectal, breast, lung, head and neck, etc since the single cells are not fixed during isolation.

According to an aspect of the present disclosure, the disclosure provides adsorbent compositions for selectively capturing, enumerating, accounting, imaging, removing and isolating marker cell for cancer such as circulating rare cells (CRCs), including CTCs and leukocytes, from the blood of a cancer patient.

In an embodiment, the adsorbent compositions comprise a substrate functionalized with a functionalizing agent that is linked through a spacer to a ligand that binds non-covalently to a marker cell for cancer.

In an embodiment, the adsorbent compositions comprise a substrate functionalized by a chemical reaction and is linked through a spacer to a ligand that binds non-covalently to a marker cell for cancer.

In an additional embodiment, the disclosure provides an adsorbent composition comprising a functionalized substrate, 1) covalently coupled or covalently linked to a spacer that is coupled to a ligand that binds non-covalently to a marker cancer cell, and 2) covalently coupled or linked to a spacer that is coupled to a ligand that binds non-covalently to leukocytes.

In any aspect or embodiment described herein, the substrate is selected from glass and hydroxylated glass, graphene oxide, silica, steel, silicon, and iron oxide.

In any aspect or embodiment described herein, the functionalizing agent is selected from (3-aminopropyl)triethoxysilane (APTES), (3-glycidyloxypropyl)trimethoxysilane (GPTMS), (3-glycidyloxypropyl)triethoxysilane (GPTES), (3-mercaptopropyl)trimethoxy silane (MPTMS), (3-mercaptopropyl)triethoxysilane (MPTES), (3-Thiocyanatopropyl)trimethoxysilane (TCPTES), and (3-Isocyanatopropyl)triethoxysilane (ICPTES).

In any aspect or embodiment described herein, the spacer is selected from 2,2′-dithiodipyridine (DTDP), 5,5′-dithiobis(2-nitrobenzoic acid) (DTNB), N-succinimidyl 3-(2-pyridyldithio)propionate) (SPDP), Poly(ethylene glycol) 2-mercaptoethyl ether acetic acid (SH-PEG-COOH), SPDP-PEG₃₆-NHS ester, 3-mercaptopropionic acid (MPA), 6-maleimidohexanoic acid, glutathione, (3-aminopropyl) triethoxysilane (APTES), (3-glycidyloxy propyl) trimethoxysilane (GPTMS), (3-glycidyloxypropyl)triethoxysilane (GPTES), (3-mercaptopropyl)trimethoxy silane (MPTMS), (3-mercaptopropyl)triethoxysilane (MPTES), (3-Thiocyanatopropyl)trimethoxysilane (TCPTES), and (3-Isocyanatopropyl)triethoxysilane (ICPTES) and cysteine.

In any aspect or embodiment described herein, the ligand that binds non-covalently to a marker cell for cancer is selected from anti-epithelial cell adhesion molecule antibody (e.g., anti EpCAM), and a protein (e.g., transferrin).

In any aspect or embodiment described herein, the ligand that non covalently binds to a marker cell for cancer is linked to the substrate through at least one linkage that is cleavable.

In any aspect or embodiment described herein, the ligand that non covalently binds to a marker cell for cancer is linked to the substrate through at least one linkage that is not cleavable.

In any aspect or embodiment described herein, the ligand that non-covalently binds to a leukocyte is linked to the substrate through a linkage that is not cleavable.

In any aspect or embodiment described herein, the ligand that non-covalently binds to a leukocyte is linked to the substrate through a linkage that is cleavable.

In any aspect or embodiment described herein, the ligand that binds to a leukocyte is an anti-CD45 antibody.

In any aspect or embodiment described herein, the spacer is linked to the functionalizing agent through a linkage that is cleavable.

In any aspect or embodiment described herein, the spacer is linked to the functionalizing agent through a linkage that is not cleavable.

In any aspect or embodiment described herein, the spacer is linked to another spacer through a linkage that is cleavable.

In any aspect or embodiment described herein, the spacer is linked to another spacer through a linkage that is not cleavable.

In any aspect or embodiment described herein, the cleavable linkage is cleaved in the presence of a stimulus selected from a reducing agent, an enzyme, and irradiation.

In any aspect or embodiment described herein, the cleavable linkage is cleaved in the presence of a reducing agent selected from dithiothreitol (DTT), tris(hydroxypropyl)phosphine (THP), and 2-mercaptoethanol (2-ME).

In any aspect or embodiment described herein, the reducing agent is dissolved in a buffer selected from sodium phosphate buffer, sodium bicarbonate buffer, tris buffered saline (TBS), and a combination thereof.

In any aspect or embodiment described herein, the cleavable linkage is cleaved in the presence of an enzyme selected from urease, and cathepsin B.

In any aspect or embodiment described herein, the cleavable linkage is cleaved in the presence of light irradiation in the frequency range 350 nm to 550 nm.

In an additional aspect, the disclosure provides methods of synthesis of adsorbent compositions as described herein for selective capture and release of marker cell for cancer.

In any aspect or embodiment described herein, the method provides a real-time, single isolated marker cell for cancer.

In any aspect or embodiment described herein, the method provides an isolated single cell that is useful in multi-omics, proteomics, identifying genetic signatures or mutations.

In any aspect or embodiment described herein, the method includes a step of monitoring the capture and release of the marker cell for cancer with at least one of manual or automated microscopy.

In any aspect or embodiment described herein, the cleavable linkages are selected from disulfide linkage, urea linkage, and tetrazole ring.

In any aspect or embodiment described herein, the non-cleavable linkages are selected from amino-alcohol linkage, and thiol-maleimide linkage.

In an additional aspect, the disclosure provides a method of isolating the single marker cell for cancer from the blood of a cancer patient comprising the steps of: 1) providing a whole blood sample (e.g., about 3 ml) from a cancer patient; 2) lysing RBCs by mixing with an RBC lysis buffer (e.g., 6 ml 1×RBC lysis buffer); 3) incubating and centrifuging the mixture (e.g., incubating for 10 min at 25° C. on a rotary shaker, and centrifuging at 500×g for 5 min at 25° C.) to form a supernatant and a pellet; 4) discarding the supernatant and resuspending the pellet in RBC lysis buffer (e.g., 1 ml 1×RBC lysis buffer); 5) incubating the resuspended mixture and centrifuging again (e.g., incubating at 25° C. for 5 min followed by centrifugation); 6) washing the pelleted cells (e.g., twice with 1×PBS (pH 7.4)); 7) staining with a staining agent; 8) incubating the cells with an adsorbent as described herein (e.g., at 25° C., 20 min) to bind a circulating tumor cell (CTC); 9) washing the adsorbent (e.g., with 1×PBS, pH 7.4); 10) incubating in the presence of a stimulus that releases the CTC; and 11) identifying and collecting the released CTC into a buffer solution (e.g., at a volume ranging from 0.2 μL to 200 μL), and isolating single CTC by pipetting while visualizing with a fluorescence microscope, and optionally storing at −80° C.

In any aspect or embodiment described herein, the stimulus that releases the CTC is selected from a reducing agent, an enzyme, irradiation, and combinations thereof.

In any aspect or embodiment described herein, the reducing agent is selected from dithiothreitol, tris (hydroxypropyl) phosphine, and combinations thereof.

In any aspect or embodiment described herein, the reducing agent is dissolved in a buffer selected from sodium phosphate buffer, sodium bicarbonate buffer, tris buffered saline (TBS), and combinations thereof.

In any aspect or embodiment described herein, the stimulus that releases the CTC is an enzyme selected from urease and cathepsin-α.

In any aspect or embodiment described herein, the stimulus that releases the CTC cells is irradiation in the range selected from 350 nm to 550 nm.

In any aspect or embodiment described herein, the method includes isolating a CTC as a single cell without fixing the cell, optionally the cell is isolated with a biomarker, such as, e.g., CK18, CD45 etc, which enable further downstream genomic processing of the single cell.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the present disclosure may be utilized in numerous combinations, all of which are expressly contemplated by the present description. These additional advantages, objects and embodiments are expressly included within the scope of the present disclosure. The publications and other materials used herein to illuminate the background of the disclosure, and in particular cases, to provide additional details respecting the practice, are incorporated by reference in their entirety for all purposes.

### BRIEF DESCRIPTION OF THE DRAWINGS

The accompanying figures, schemes and/or drawings which are incorporated into, and form a part of the specification, illustrate several embodiments of the present disclosure and, together with the description, serve to explain the principles of the disclosure. The figures, schemes and/or drawings are only for the purpose of illustrating embodiments of the disclosure and are not to be construed as limiting the disclosure. Further objects, features and advantages of the disclosure will become apparent from the detailed description taken in conjunction with the accompanying figures showing illustrative embodiments of the present disclosure, in which:

FIG. 1. Illustrates a generalized scheme for the design of cell capture and release compositions/methods as described herein.

FIG. 2. Illustrates an exemplary molecular design scheme for a cell capture adsorbent composition as described herein, useful to isolate single cell(s).

FIG. 3. Illustrates a generalized process as described herein for the selective capture and release of marker cells for cancer from peripheral blood.

FIG. 4. Illustrates an exemplary process for the selective capture and release of marker cells for cancer from peripheral blood as described herein.

FIG. 5. Illustrates a generalized process as described herein for the selective capture and release of marker cells for cancer from pre-processed blood.

FIG. 6. Illustrates an exemplary process for the selective capture and release of marker cells for cancer from pre-processed blood as described.

FIG. 7. Illustrates an exemplary conjugation of hydroxylated glass beads with (3-mercaptopropyl)trimethoxysilane spacer.

FIG. 8. Illustrates an exemplary conjugation of 2,2′-dithiodipyridine (DTDP) spacer with thiol bearing glass beads.

FIG. 9. Illustrates an exemplary conjugation of 3-mercaptopropionic acid (MPA) spacer with glass beads bearing disulfide linkages.

FIGS. 10A, 10B, and 10C. Illustrates an exemplary conjugation of glass beads bearing terminal carboxylic group with A) anti-EpCAM, B) anti-CD45, and C) transferrin.

FIG. 11. Illustrates an exemplary conjugation of 5,5′-dithiobis (2-nitrobenzoic acid) (DTNB) spacer with thiol bearing glass beads.

FIG. 12. Illustrates an exemplary synthesis of glass beads bearing terminal carboxylic acid groups.

FIGS. 13A, 13B, and 13C. Illustrates an exemplary conjugation of glass beads bearing terminal carboxylic acid groups with A) anti-EpCAM, B) anti-CD45, and C) transferrin.

FIG. 14. Illustrates an exemplary conjugation of 6-maleimidohexanoic acid spacer with thiol bearing glass beads.

FIGS. 15A, 15B, and 15C. Illustrates an exemplary conjugation of glass beads bearing terminal carboxylic acid groups with A) anti-EpCAM, B) anti-CD45, and C) transferrin.

FIG. 16. Illustrates an exemplary conjugation of mercapto-PEG-carboxylic acid spacer with glass beads bearing disulfide linkages.

FIGS. 17A, 17B, and 17C. Illustrates an exemplary conjugation of glass beads bearing terminal carboxylic acid groups with A) anti-EpCAM antibody, B) anti-CD45 antibody and C) transferrin.

FIG. 18. Illustrates an exemplary synthesis of glass beads bearing terminal carboxylic acid groups.

FIGS. 19A, 19B, and 19C. Illustrates an exemplary conjugation of glass beads bearing terminal carboxylic acid groups with A) anti-EpCAM antibody, B) anti-CD45 antibody and C) transferrin.

FIG. 20. Illustrates an exemplary functionalization of iron oxide nanoparticles with (3-mercaptopropyl) trimethoxysilane.

FIG. 21. Illustrates an exemplary conjugation of 2,2′-dithiodipyridine (DTDP) spacer with thiol bearing iron oxide nanoparticles.

FIG. 22. Illustrates an exemplary conjugation of 3-mercaptopropionic acid (MPA) spacer with iron oxide nanoparticles bearing disulfide linkage.

FIGS. 23A, 23B, and 23C. Conjugation of iron oxide nanoparticles bearing terminal carboxylic acid group with A) anti-EpCAM antibody, B) anti-CD45 antibody and C) transferrin.

FIG. 24. Illustrates an exemplary conjugation of 5,5′-dithiobis (2-nitrobenzoic acid) (DTNB) spacer with thiol bearing iron oxide nanoparticles.

FIG. 25. Illustrates an exemplary conjugation of 3-mercaptopropionic acid (MPA) spacer with disulfide bearing iron oxide nanoparticles.

FIGS. 26A, 26B, and 26C. Illustrates an exemplary conjugation of iron oxide nanoparticles bearing terminal carboxylic acid groups with A) anti-EpCAM antibody, B) anti-CD45 antibody and C) transferrin.

FIG. 27. Illustrates an exemplary conjugation of 6-maleimidohexanoic acid spacer with thiol bearing iron oxide nanoparticles.

FIGS. 28A, 28B, and 28C. Conjugation of iron oxide nanoparticles bearing terminal carboxylic acid groups with A) anti-EpCAM antibody, B) anti-CD45 antibody and C) transferrin.

FIG. 29. Illustrates an exemplary conjugation of graphene oxide with (3-mercaptopropyl)trimethoxysilane.

FIG. 30. Illustrates an exemplary conjugation of 2,2′-dithiodipyridine (DTDP) spacer with thiol bearing graphene oxide nanoparticles.

FIG. 31. Illustrates an exemplary conjugation of 3-mercaptopropionic acid (MPA) spacer with graphene oxide nanoparticles bearing disulfide linkages.

FIGS. 32A, 32B, and 32C. Conjugation of graphene oxide nanoparticles bearing terminal carboxylic acid groups with A) anti-EpCAM antibody, B) anti-CD45 antibody and C) transferrin.

FIG. 33. Illustrates an exemplary conjugation of 5,5′-dithiobis (2-nitrobenzoic acid) (DTNB) spacer with thiol bearing graphene oxide nanoparticles.

FIG. 34. Illustrates an exemplary conjugation of 3-mercaptopropionic acid (MPA) spacer with graphene oxide nanoparticles bearing disulfide linkages.

FIGS. 35A, 35B, and 35C. Conjugation of graphene oxide nanoparticles bearing terminal carboxylic acid groups with A) anti-EpCAM antibody, B) anti-CD45 antibody and C) transferrin.

FIG. 36. Illustrates an exemplary conjugation of 6-maleimidohexanoic acid spacer with thiol bearing graphene oxide nanoparticles.

FIGS. 37A, 37B, and 37C. Illustrates an exemplary conjugation of graphene oxide nanoparticles bearing terminal carboxylic acid groups with A) anti-EpCAM antibody, B) anti-CD45 antibody and C) transferrin.

FIG. 38. Illustrates an exemplary functionalization of glass beads using two different silanes.

FIG. 39. Illustrates an exemplary conjugation of 2,2′-dithiodipyridine (DTDP) spacer with thiol group of glass beads bearing two different silanes.

FIGS. 40A and 40B. Illustrates an exemplary thiolation of A) anti-EpCAM and B) anti-CD45 antibody with 2-iminothiolane.

FIG. 41. Illustrates an exemplary synthesis of adsorbent bearing anti-EpCAM antibody conjugated through cleavable linkage and anti-CD45 antibody conjugated through non-cleavable linkage.

FIG. 42. Illustrates an exemplary synthesis of adsorbent bearing anti-EpCAM antibody conjugated through non-cleavable linkage and anti-CD45 antibody conjugated through cleavable linkage.

FIGS. 43A and 43B. Illustrates an exemplary conjugation of N-succinimidyl-3-(2-pyridyldithio) propionate (SPDP) spacer with A) anti-EpCAM and B) anti-CD45 antibody.

FIG. 44. Illustrates an exemplary synthesis of adsorbent bearing anti-EpCAM antibody conjugated through cleavable linkage and anti-CD45 antibody conjugated through non-cleavable linkage.

FIG. 45. Illustrates an exemplary synthesis of adsorbent bearing conjugated anti-EpCAM antibody through non-cleavable linkage and conjugated anti-CD45 antibody through cleavable linkage.

FIG. 46. Illustrates an exemplary conjugation of hydroxylated glass beads with (3-isocyanatopropyl) triethoxysilane (ICPTES).

FIGS. 47A, 47B, and 47C. Conjugation of isocyanate bearing glass beads with A) anti-EpCAM antibody, B) anti-CD45 antibody and C) transferrin.

FIG. 48. Illustrates an exemplary conjugation of glass beads with (3-Thiocyanatopropyl) triethoxysilane (TCPTES).

FIG. 49. Illustrates an exemplary synthesis of adsorbent bearing terminal carboxylic acid groups.

FIGS. 50A, 50B, and 50C. Illustrates an exemplary conjugation of adsorbent bearing terminal carboxylic acid groups with A) anti-EpCAM antibody, B) anti-CD45 antibody and C) transferrin.

FIGS. 51A and 51B. Illustrates an exemplary conjugation of SPDP-PEG₃₆-NHS ester spacer with A) anti-EpCAM and B) anti-CD45 antibody.

FIG. 52. Illustrates an exemplary synthesis of adsorbent bearing anti-EpCAM antibody conjugated through cleavable linkage and anti-CD45 antibody conjugated through non-cleavable linkage.

FIG. 53. Illustrates an exemplary synthesis of adsorbent bearing anti-EpCAM antibody conjugated through non-cleavable linkage and anti-CD45 antibody conjugated through cleavable linkage.

FIG. 54. Illustrates an exemplary conjugation of glutathione spacer with glass beads bearing disulfide linkage.

FIGS. 55A, 55B, and 55C. Illustrates an exemplary conjugation of adsorbent bearing terminal carboxylic acid groups with A) anti-EpCAM antibody, B) anti-CD45 antibody and C) transferrin.

FIG. 56. Illustrates an exemplary dithiothreitol (DTT) catalysed cleavage of disulfide linkage on glass beads.

FIG. 57. Illustrates time-dependent tris(hydroxypropyl)phosphine (THP) catalysed cleavage of disulfide linkage.

FIG. 58. Illustrates selective capture and release of human breast cancer cells (MCF-7) using adsorbent bearing transferrin.

FIGS. 59A, 59B, and 59C. Illustrates selective capture, release and collection of single circulating tumor cells (CTCs) using adsorbent bearing transferrin.

FIGS. 60A, 60B, and 60C. Illustrates eluting non-specifically bound leukocytes using phosphate buffer saline.

FIG. 61. Illustrates selective capture and release of spiked MCF-7 cells from the blood of a healthy individual using adsorbent bearing transferrin.

FIG. 62. Illustrates selective capture and release of CTCs using adsorbent bearing anti-EpCAM antibody.

FIG. 63. Illustrates capture and selective release of circulating tumor cells (CTCs) using adsorbent bearing anti-EpCAM antibody through cleavable linkage and anti-CD45 antibody through non-cleavable linkage.

FIG. 64. Illustrates capture and selective release of leukocytes using adsorbent bearing anti-CD45 antibody through cleavable linkage and anti-EpCAM antibody through non-cleavable linkage.

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

The origin of diversity in cellular structure and function stems from the heterogeneity at the molecular level. Therefore, it is necessary to comprehend the nature of cellular heterogeneity for investigating normal differentiation, development of living cells, and to understand aberrant behaviour in pathophysiological context. Many biological processes are being profiled at single cell level to understand neuron heterogeneity, early embryonic development, and identify uncultivatable microbes. These lead to insights into evolution of many diseases including cancer. Considering the complex nature of cancer as a disease, studying heterogeneity in tumor cells is useful to determine the clonal population, metastatic behaviour and occurrence of the cancer stem cells. These aspects of cancer cells in tumor milieu can eventually define the choice of anticancer therapy. Therefore, investigating cancer cells at single-cell level is becoming imperative and technological development in this direction would enable providing novel clinical choices to offer personalized treatments. Technological advancements in multi-omics platform have made it possible to identify how disease-resulting gene-alterations dysregulate protein-protein interactions within human cells harbouring the mutation. However, the output from these platforms is significantly biased towards the input information. Additionally, any clinically actionable information on nature of the tumor microenvironment in metastatic settings is not obtained. This problem can be greatly minimized by isolating single tumor cells and performing multi-omics analysis. However, isolating single tumor cells from the site of tissue is technologically challenging. Moreover, considering the biological heterogeneity of tumor, it is extremely difficult to choose the ‘right’ cell for isolation from a mass of tumor and perform single cell sequencing.

Importantly, multi-omics analysis is not feasible on temporal scale due to the limitations on obtaining tissue biopsies longitudinally. Circulating tumor cells (CTCs) have emerged as a novel tool for predicting cancer prognosis and the disease outcome in clinical settings. CTCs are likely to display similar spatial and temporal heterogeneity and plasticity as the primary tumor. CTCs can reveal the true nature of necrotic tissue in ‘real time’ for genomic evolution from its whole genome amplification. CTCs and their comprehensive genomic panel are more relevant for actionable, precision, and personalised therapies, drug development and drug discovery phases to mapping the heterogenous circulating tumor DNA (CTDNA) analysis using blood or tissue. Therefore, isolating CTCs from blood offers a viable and practical alternative to performing multi-omics analysis on individual cancer cells. Single cell analysis can be performed by isolating a whole cell or cell-specific nuclei or organelles. Many therapy decisions, including immuno-oncology and targeted therapies could improve the progression free survival or overall survival.

Where a range of values is provided, it is understood that unless the context clearly dictates otherwise, each intervening value, to the tenth of the unit of the lower limit between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention unless the context indicates otherwise.

The following terms are used to describe the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting of the invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It is well-established that at the origin of primary cancer, the tissue sheds the CTCs. CTCs because of their plasticity enter in peripheral blood circulation which further leads to clinical manifestations. Furthermore, these cells disseminate and circulate and colonize to form distant metastasis.

CTCs when in the form of group as ‘cell clusters’ are known to be up to 50 times more aggressive in translating the distant tumor metastasis compared to CTCs alone. The presence of CTCs in blood circulation predicts and is associated with the progression of disease, short survival, failure to respond to treatment, and can aid in real-time monitoring of the patient for minimal residual disease. However, it is practically impossible to target CTCs and their destruction in blood circulation with sub-cellular cytotoxic drug concentrations and by any other therapy including radiation.

CTCs and other biomarkers such as cell-free nucleic acids (CfDNA), circulating tumor DNA (CtDNA), mutations like Epidermal growth factor receptor (EGFR), BReast CAncer gene (BRCA), and prostate serum antigen (PSA) can be used along with other tools, such as CT/PET imaging, biopsy, histopathological staining, mammography.

Thus, it is desirable in the art of cancer therapy to eliminate CTCs from patient's whole blood to reduce and eventually prevent metastatic progression and increase the patient's overall survival (Pantel, K., et al. Nat Rev Clin Oncol. 2019, 16, 409-424; Scarberry, K. E. et al. Nanomedicine. 2011, 6 1, 69-78; Azarin S., et al. Nat Comm. 2015, 6, 8094). (Cohen et al. J. Clin. Oncol. 2008, 26, 3213-3221).

Similarly, the removal of other cancer-causing entities, for example, cell-free nucleic acids (CfDNA), cancer cells associated with nucleic acids (CtDNA), exosomes, and chemical entities such as chemo drugs would lead to increase the progression-free survival and overall survival of cancer patients.

Indeed, the identification and characterization of CTCs for cancer phenotype, genotype, and the organ of origin will lead to design of drugs which are better targeted. Also, the development of medical devices constituting non-hemolytic adsorbent compositions will be beneficial for cancer treatment for both early as well late-stage cancers.

The present disclosure surprisingly and unexpectedly provides compositions and methods capable of capturing, purifying and isolating CTCs from the blood of a cancer patient. It is expressly contemplated that any of the above elements can be combined with any of the other elements described herein in any combination.

Compositions

In an aspect, the present disclosure provides adsorbent compositions for selectively capturing, enumerating, accounting, imaging, removing and isolating marker cell for cancer such as circulating rare cells (CRCs), including CTCs and leukocytes, from a biological sample of a subject or patient, e.g., a fluid such as blood, serum, spinal fluid, urine, etc. In certain embodiments, the subject or patient is a cancer patient

In an embodiment, the adsorbent compositions comprise a substrate functionalized with a functionalizing agent that is linked through a spacer to a ligand that binds non-covalently to a marker cell for cancer. As used herein, a “functionalizing agent” can mean a molecule that binds to the substrate non-covalently at one end and is covalently linked to a spacer at the other end.

In any aspect or embodiment described herein, the substrate is selected from glass and hydroxylated glass, graphene oxide, silica, steel, silicon, and iron oxide.

In any aspect or embodiment described herein, the substrate is covalently coupled or linked to the spacer through a functionalizing agent; and/or the spacer is covalently coupled or linked to the marker specific ligand.

As used herein, the term “functionalization” can refer to the process of creating functional groups on the surface of a substrate, so that the spacer can be conjugated to the functional group. Functionalization can be carried out using a functionalizing agent or a chemical reaction. The functionalization of the substrate can be performed by any method known in the art, including those described in co-pending US Patent Publication 20210106742, which is incorporated herein by reference in its entirety.

As used herein, the term “spacer” can mean a molecule having a functional group at both ends, which is conjugated to (a) A functionalized substrate, functionalizing agent and/or a spacer at one end and (b) a spacer or a marker specific ligand at another end. A given molecule may act either as a spacer or a functionalizing agent depending on the choice of the substrate.

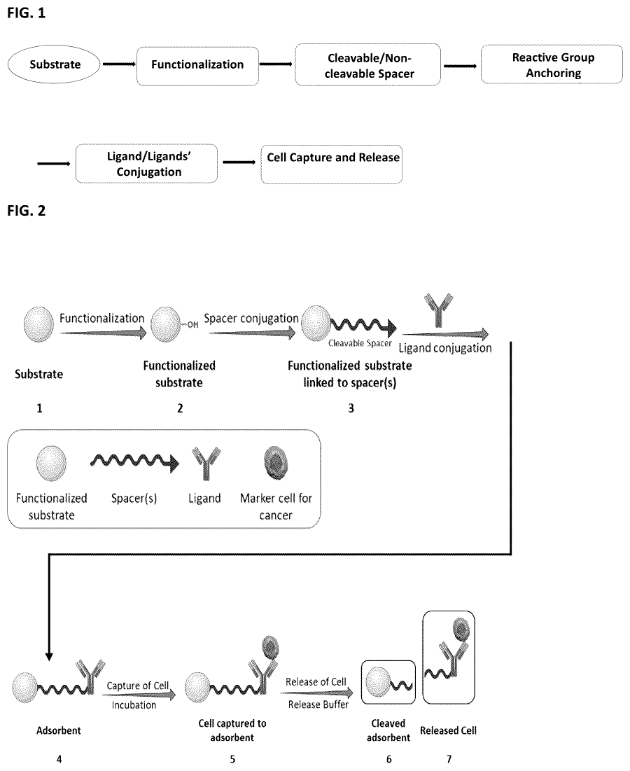

In any aspect or embodiment described herein, the substrate is functionalized and conjugated with at least one spacer and then with a ligand that binds to 1) a marker cell for cancer, and/or 2) a leukocyte to yield an adsorbent. The cells expressing appropriate sites for the respective ligand(s), interact selectively with the ligand and are captured on the adsorbent surface (See FIG. 1).

In an additional embodiment, the disclosure provides an adsorbent composition comprising a functionalized substrate, 1) covalently coupled or linked to a spacer that is coupled to a ligand that binds non-covalently to a marker cancer cell, and/or 2) covalently coupled or linked to a spacer that is coupled to a ligand that binds non-covalently to leukocytes.

In any aspect or embodiment described herein, the substrate is functionalized so that appropriate functional group is available to link it with the spacer molecule through a cleavable or non-cleavable linkage. As described herein, a covalent linkage that is cleavable is one that is cleaved in the presence of a specific stimulus. A covalent linkage that is non-cleavable is one that is not cleaved in the presence of the same specific stimulus.

In certain embodiments, the composition can include more than one spacer. When more than one spacer is present, at least one linkage between two spacers is cleavable, which leads to the release of the marker cell for cancer. (See FIG. 2).

In any aspect or embodiment described herein, the functionalizing agent is selected from (3-aminopropyl)triethoxysilane (APTES), (3-glycidyloxypropyl)trimethoxysilane (GPTMS), (3-glycidyloxypropyl)triethoxysilane (GPTES), (3-mercaptopropyl)trimethoxy silane (MPTMS), (3-mercaptopropyl)triethoxysilane (MPTES), (3-Thiocyanatopropyl)trimethoxysilane (TCPTES), and (3-Isocyanatopropyl)triethoxysilane (ICPTES).

In any aspect or embodiment described herein, the spacer is selected from 2,2′-dithiodipyridine (DTDP), 5,5′-dithiobis(2-nitrobenzoic acid) (DTNB), N-succinimidyl 3-(2-pyridyldithio)propionate) (SPDP), Poly(ethylene glycol) 2-mercaptoethyl ether acetic acid (SH-PEG-COOH), SPDP-PEG₃₆-NHS ester, 3-mercaptopropionic acid (MPA), 6-maleimidohexanoic acid, glutathione, (3-aminopropyl) triethoxysilane (APTES), (3-glycidyloxy propyl) trimethoxysilane (GPTMS), (3-glycidyloxypropyl)triethoxysilane (GPTES), (3-mercaptopropyl)trimethoxy silane (MPTMS), (3-mercaptopropyl)triethoxysilane (MPTES), (3-Thiocyanatopropyl)trimethoxysilane (TCPTES), and (3-Isocyanatopropyl)triethoxysilane (ICPTES) and cysteine.

In any aspect or embodiment described herein, the ligand that binds non-covalently to a marker cell for cancer is selected from anti-epithelial cell adhesion molecule antibody (e.g., anti EpCAM), and a protein (e.g., transferrin).

In any aspect or embodiment described herein, the ligand that non covalently binds to a marker cell for cancer is linked to the substrate through at least one linkage that is cleavable.

In any aspect or embodiment described herein, the ligand that non-covalently binds to a leukocyte is linked to the substrate through a linkage that is not cleavable.

In any aspect or embodiment described herein, the ligand that binds to a leukocyte is an anti-CD45 antibody.

In any aspect or embodiment described herein, the spacer is linked to the functionalizing agent through a linkage that is cleavable.

In any aspect or embodiment described herein, the spacer is linked to the functionalizing agent through a linkage that is not cleavable.

In any aspect or embodiment described herein, the spacer is linked to another spacer through a linkage that is cleavable.

In any aspect or embodiment described herein, the spacer is linked to another spacer through a linkage that is not cleavable.

In any aspect or embodiment described herein, the cleavable linkage is cleaved in the presence of a stimulus selected from a reducing agent, an enzyme, and irradiation.

In any aspect or embodiment described herein, the cleavable linkage is cleaved in the presence of a reducing agent selected from dithiothreitol (DTT), tris(hydroxypropyl)phosphine (THP), and 2-mercaptoethanol (2-ME).

In any aspect or embodiment described herein, the reducing agent is dissolved in a buffer selected from sodium phosphate buffer, sodium bicarbonate buffer, tris buffered saline (TBS), and a combination thereof.

In any aspect or embodiment described herein, the cleavable linkage is cleaved in the presence of an enzyme selected from urease, and cathepsin B.

In any aspect or embodiment described herein, the cleavable linkage is cleaved in the presence of light irradiation in the frequency range 350 nm to 550 nm.

Methods

In an additional aspect, the disclosure provides methods of synthesis of an adsorbent composition as described herein for selective capture and release of marker cell for cancer.

In certain embodiments, the method comprises the steps of providing a substrate; and functionalizing the substrate with a functionalizing agent; coupling a spacer to the functionalizing agent; and coupling a target specific ligand to the spacer to form an adsorbent composition for selectively capturing, enumerating, accounting, imaging, removing and isolating a marker cell for cancer, e.g., a circulating rare cell (CRC), including a CTC and a leukocyte.

In an additional aspect, the disclosure provides methods of isolating the single marker cell for cancer from the blood of a cancer patient comprising the steps of: 1) providing a whole blood sample (e.g., about 3 ml) from a cancer patient; 2) lysing RBCs by mixing with an RBC lysis buffer (e.g., 6 ml 1×RBC lysis buffer); 3) incubating and centrifuging the mixture (e.g., incubating for 10 min at 25° C. on a rotary shaker, and centrifuging at 500×g for 5 min at 25° C.) to form a supernatant and a pellet; 4) discarding the supernatant and resuspending the pellet in RBC lysis buffer (e.g., 1 ml 1×RBC lysis buffer); 5) incubating the resuspended mixture and centrifuging again (e.g., incubating at 25° C. for 5 min followed by centrifugation); 6) washing the pelleted cells (e.g., twice with 1×PBS (pH 7.4)); 7) staining with a staining agent; 8) incubating the cells with an adsorbent as described herein (e.g., at 25° C., 20 min) to bind a circulating tumor cell (CTC); 9) washing the adsorbent (e.g., with 1×PBS, pH 7.4); 10) incubating in the presence of a stimulus that releases the CTC; and 11) identifying and collecting the released CTC into a buffer solution (e.g., at a volume ranging from 0.2 μL to 200 μL), and isolating single CTC by pipetting while visualizing with a fluorescence microscope, and optionally storing at −80° C.

In any aspect or embodiment described herein, the stimulus that releases the CTC is selected from a reducing agent, an enzyme, irradiation, and combinations thereof.

In any aspect or embodiment described herein, the reducing agent is selected from dithiothreitol, tris (hydroxypropyl) phosphine, and combinations thereof.

In any aspect or embodiment described herein, the reducing agent is dissolved in a buffer selected from sodium phosphate buffer, sodium bicarbonate buffer, tris buffered saline (TBS), and combinations thereof.

In any aspect or embodiment described herein, the stimulus that releases the CTC is an enzyme selected from urease and cathepsin-B.

In any aspect or embodiment described herein, the stimulus that releases the CTC is irradiation in the range selected from 350 nm to 550 nm.

In any aspect or embodiment described herein, the method includes isolating a CTC as a single cell without fixing and further staining the cell for biomarkers, such as CK18, CD45 etc, which enable further downstream genomic processing of the single cell.

In any aspect or embodiment described herein, the method provides a real-time, single isolated marker cell for cancer.

In any aspect or embodiment described herein, the method provides an isolated single cell that is useful in multi-omics, proteomics, identifying genetic signatures or mutations.

In any aspect or embodiment described herein, the method includes a step of monitoring the capture and release of the marker cell for cancer with at least one of manual or automated microscopy.

In any aspect or embodiment described herein, the cleavable linkages are selected from disulfide linkage, urea linkage, and tetrazole ring.

In any aspect or embodiment described herein, the non-cleavable linkages are selected from amino-alcohol linkage, and thiol-maleimide linkage.

Selective Capture, Release, and Detection of Cancer Cell(s) from Peripheral Blood Sample

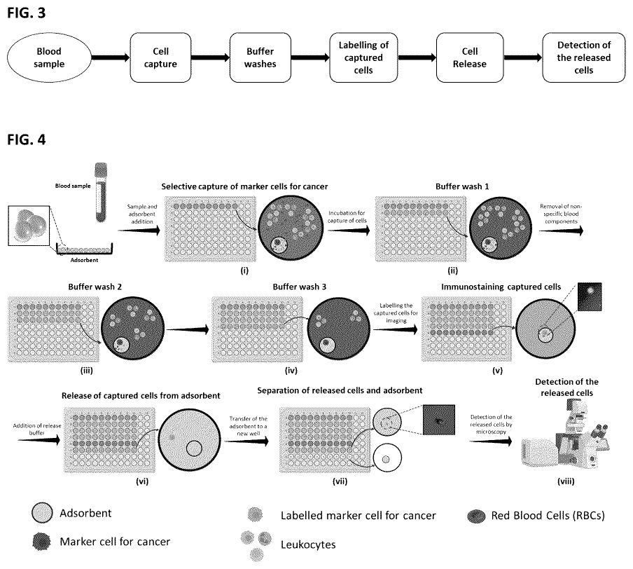

With reference to FIGS. 3 and 4. Blood sample was mixed with an adsorbent composition as described herein to selectively capture the cancer cell(s). After mixing and incubating with the adsorbent, non-specifically bound blood components were washed in sequential manner with wash buffer (Step ii to iv). This step effectively removed the weakly bound blood components without affecting the stability of selectively bound cell(s).

The cell(s) bound to the adsorbent were fluorescently labelled for their identification using fluorescence imaging (Step v). Later, the adsorbent bearing cell(s) was incubated with the release buffer (Step vi). After incubation, captured cell(s) were released in the surrounding solution (Step vii), and identified using a fluorescence microscope (Step viii).

Selective capture, release, and detection of cancer cell(s) from processed peripheral blood sample.

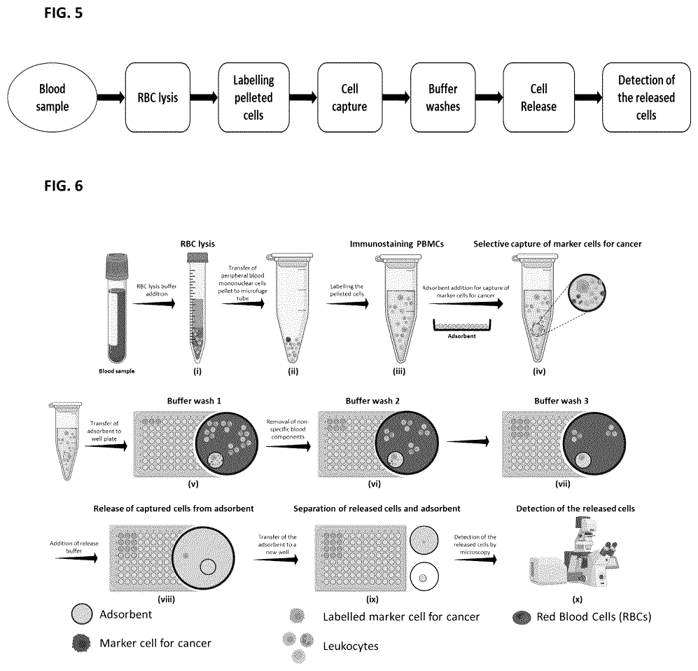

With reference to FIGS. 5 and 6. Blood sample was mixed with RBC lysis buffer to remove the RBCs from the blood sample (Step i). The RBCs were separated by centrifugation. The remainder was resuspended in 1× phosphate buffered saline (PBS), pH 7.4 buffer and held at room temperature (Step ii). The resuspended cells, including leukocytes and cancer cells were then immunostained (Step iii). The stained cells were mixed and incubated with an adsorbent composition as described herein for the selective capture of the specific cells present in the blood (Step iv). The cells bound to an adsorbent were distributed to a 96-well plate such that each well would receive equal number of beads and equal volume of the buffer. The adsorbent in each well was washed and transferred in subsequent wells for sequential washings with the 1×PBS (pH 7.4) thereafter (Step v). To remove non-specifically bound cells and blood components, the adsorbent was washed repeatedly with the wash buffer in a sequential manner (Steps vi and vii). The adsorbent was then mixed and incubated in presence of the release stimuli to facilitate the release of selectively captured cancer cells (Step viii). The released cancer cells, which were specifically labelled (Step iii) were subsequently detected using an automated imaging workflow and a computer-controlled fluorescence microscope.

Synthesis of selective adsorbents bearing cleavable as well as non-cleavable linkages.

### EXAMPLES

The examples below are to be regarded as illustrative in nature and do not limit the scope of the disclosure in any manner.

### Example 1

### Preparation of Clean Glass Beads

10 g glass beads (diameter 2 mm) were immersed in 50 ml 5% v/v hydrochloric acid in milli-Q water and then stirred for 12 h at room temperature. The supernatant was decanted. The glass beads were washed thrice with 50 ml milli-Q water followed by 50 ml acetone. They were then dried in hot air oven at 80° C. for 1 hour and stored at room temperature in a dry place.

### Example 2

Preparation of hydroxylated glass beads using piranha solution (mixture of sulfuric acid (H₂SO₄) and hydrogen peroxide (H₂O₂) (3:1)). 9 g cleaned glass beads of Example 1 were immersed in 75 ml (98%) sulphuric acid. 25 ml (30% v/v in water) hydrogen peroxide was then added drop-wise to the reaction medium (Piranha solution) and heated at 85° C. for 2 h. The reaction medium was then allowed to cool to room temperature. The hydroxylated glass beads were washed three times with 50 ml milli-Q water, then with 50 ml acetone, dried in oven at 80° C. for 1 hour and stored at room temperature in a dry place.

### Example 3

### Conjugation of Hydroxylated Glass Beads with (3-Mercaptopropyl)Trimethoxysilane

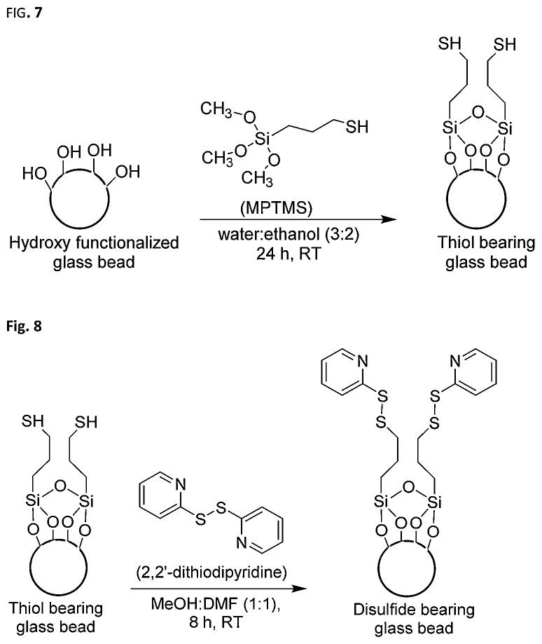

A solution of 5% (3-mercaptopropyl)trimethoxysilane (MPTMS) in water:ethanol (3:2 v/v) was hydrolyzed for 12 h. 7 g hydroxylated glass beads of Example 2 were immersed in the above solution in a petri plate and shaken for 12 h over rocker shaker at room temperature. These glass beads were then washed thrice with 50 ml milli-Q water followed by 50 ml acetone and dried in oven at 80° C. for 1 h. Thiolated glass beads were stored at 4° C. (See FIG. 7). The Fourier transform infrared (FTIR) spectroscopy peaks at 2925, 2880-2847 cm′ for asymmetric and symmetric —CH₂ stretching, and 2548 cm′ for —S—H— stretching, respectively confirmed the presence of thiol group on the glass surface. The peaks at 1440-700 cm⁻¹ represent the silica skeleton (—Si—O—Si—) (silane functionality) confirming the silanization on glass surface.

### Example 4

### Conjugation of 2,2′-Dithiodipyridine (DTDP) Spacer with Thiol Bearing Glass Beads

1 mM 2,2′-dithiodipyridine solution was prepared in methanol:dimethylformamide (MeOH:DMF) (1:1 v/v). 2.5 g thiol bearing glass beads of example 3 were immersed in 30 ml of above solution in a petri plate and shaken for 8 h over rocker shaker at room temperature. These glass beads were then washed thrice with 30 ml methanol, dried at 80° C. in hot air oven for 1 h and stored at 4° C. (See FIG. 8). The Fourier transform infrared (FTIR) spectroscopy peaks at 2923, 2882-2853 cm′ for asymmetric and symmetric —CH₂ stretching confirmed the presence of —CH₂—CH₂— group on glass surface. The disappearance of peak at 2545 cm⁻¹ for —S—H— stretching confirmed the formation of disulfide bond (—S—S—). The FTIR spectra in the range 1438-700 cm⁻¹ represent the silica skeleton (—Si—O—Si—) (silane functionality) confirming the silanization on glass surface.

### Example 5

### Conjugation of 3-Mercaptopropionic Acid (MPA) Spacer with Glass Beads Bearing Disulfide Linkages

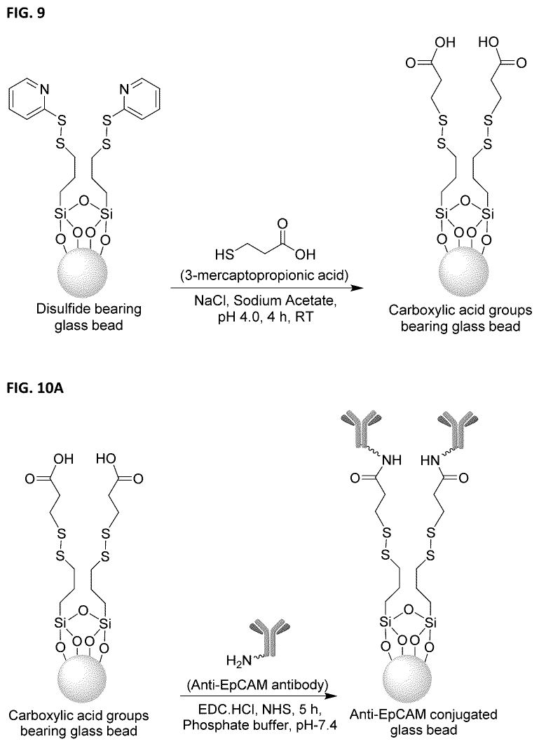

25 mM 3-mercaptopropionic acid (MPA) solution was prepared in pH 4.0 buffer solution (1 M sodium chloride (NaCl), 0.1 M sodium acetate). 1 g glass beads of example 4 were incubated in 20 ml of above solution for 4 h over rocker shaker at room temperature in a petri plate. These glass beads were washed thrice with 20 ml milli-Q water followed by 20 ml ethanol, dried in oven at 80° C. for 1 h and stored at 4° C. (See FIG. 9). The Fourier transform infrared (FTIR) spectroscopy peaks at 2914 and 2849 cm⁻¹ for asymmetric and symmetric —CH₂ stretching confirmed the presence of —CH₂—CH₂— group on glass surface. The disappearance of the peak at 2545 cm⁻¹ for —S—H— stretching confirmed the formation of disulfide bond (—S—S—). FTIR peak at 1711 cm⁻¹ (stretch) corresponds to carboxylic —C═O functional group. The FTIR spectra in the range 1442-700 cm⁻¹ represent the silica skeleton (—Si—O—Si—) (silane functionality) confirming the silanization on glass surface.

### Example 6

### Conjugation of Glass Beads Bearing Terminal Carboxylic Acid Group with Anti-EpCAM Antibody, Anti-CD45 Antibody and Transferrin

200 mg glass beads of example 5 were added to a solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. Reaction mixture was decanted and glass beads were washed with 1 ml phosphate buffer (pH 7.4) thrice. These glass beads were added to 2 ml phosphate buffer (pH 7.4), followed by addition of 10 μg anti-EpCAM antibody. The reaction mixture was kept for 4 h over rocker shaker at room temperature in a petri plate. These glass beads were washed with 2 ml phosphate buffer (pH 7.4) thrice, and stored in 2 ml PBS (pH 7.4) at 4° C.

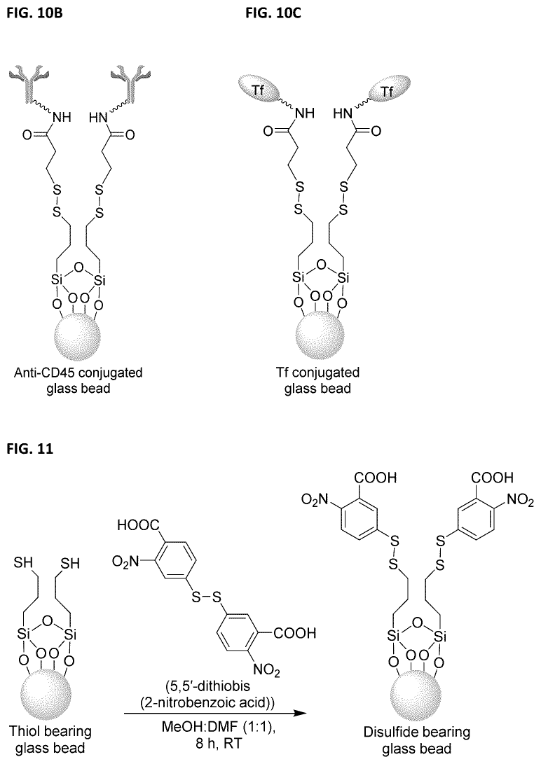

Anti-CD45 antibody and transferrin were similarly conjugated under identical reaction conditions. The structures of the conjugates are shown in FIGS. 10A, 10B, and 10C respectively. The Fourier transform infrared (FTIR) spectroscopy peaks at 2916 and 2851 cm⁻¹ for asymmetric and symmetric CH₂ stretching confirmed the presence of —CH₂—CH₂— group on glass surface. The disappearance of peak at 2545 cm⁻¹ for —S—H— stretching reflected the formation of disulfide bond (—S—S—). FTIR peak at 1709 cm⁻¹ (stretch) corresponding to amidic —C═O functional group. The FTIR spectra in the range 1440-700 cm⁻¹ represent the silica skeleton (—Si—O—Si—) (silane functionality) confirming the silanization of glass surface.

### Example 7

### Conjugation of (5,5′-Dithiobis (2-Nitrobenzoic Acid)) (DTNB) Spacer with Thiol Bearing Glass Beads

1 mM (5,5′-dithiobis (2-nitrobenzoic acid)) (DTNB) solution was prepared in MeOH:DMF (1:1 v/v). 2.5 g glass beads of example 3 were immersed in 30 ml of above solution in a petri plate and shaken for 8 h over rocker shaker at room temperature. These glass beads bearing disulfide linkages were washed thrice with 30 ml methanol, dried at 80° C. in hot air oven for 1 h. and stored at 4° C. (See FIG. 11).

### Example 8

### Synthesis of Glass Beads Bearing Terminal Carboxylic Acid Groups

25 mM3-mercaptopropionic acid (MPA) solution was prepared in a pH 4.0 buffer solution (1 M sodium chloride (NaCl), 0.1 M sodium acetate). 1 g glass beads of example 7 were incubated in 20 ml of above solution for 4 h over rocker shaker at room temperature in a petri plate. These glass beads bearing terminal carboxylic acid groups were washed thrice with 20 ml milli-Q water, then with 20 ml ethanol, dried in oven at 80° C. for 1 h. and stored at 4° C. (See FIG. 12).

### Example 9

### Conjugation of Glass Beads Bearing Terminal Carboxylic Acid Groups with Anti-EpCAM Antibody, Anti-CD45 Antibody and Transferrin

200 mg glass beads of example 8 were added in a solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. Reaction mixture was decanted and glass beads were washed with 1 ml phosphate buffer (pH 7.4) thrice. These glass beads were added to 2 ml phosphate buffer (pH 7.4), followed by addition of 10 μg anti-EpCAM antibody. The reaction mixture was kept for 4 h over rocker shaker at room temperature in a petri plate. These glass beads were washed with 2 ml phosphate buffer pH 7.4 thrice, and stored in 2 ml PBS pH 7.4 at 4° C.

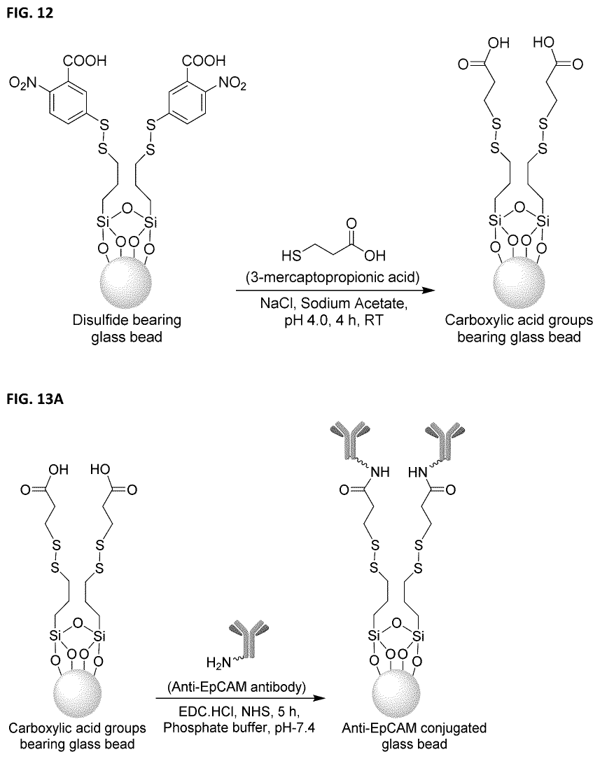

Anti-CD45 antibody and transferrin were similarly conjugated under identical reaction conditions. The structures of these conjugates are shown in FIGS. 13A, 13B and 13C respectively.

### Example 10

### Conjugation of 6-Maleimidohexanoic Acid Spacer with Thiol Bearing Glass Beads

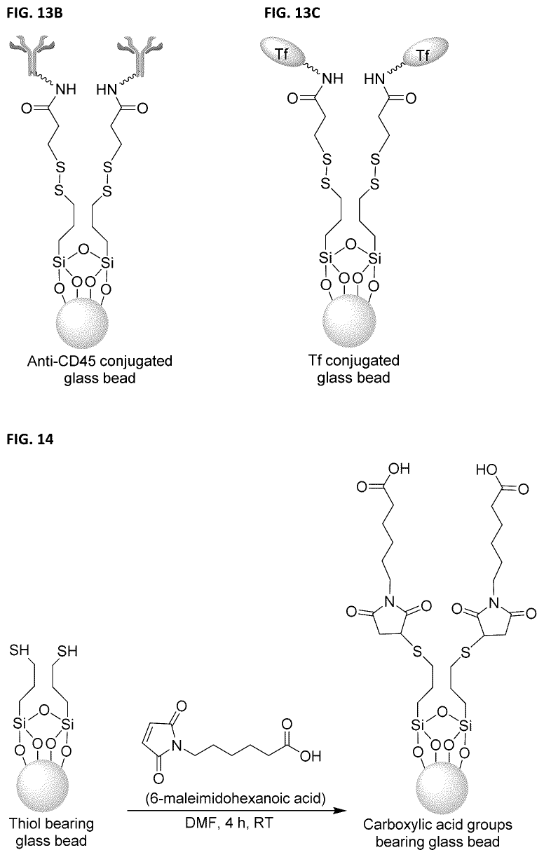

25 mM 6-maleimidohexanoic acid solution was prepared in dimethyl formamide (DMF). 1 g glass beads of example 3 were incubated in above solution for 4 h over rocker shaker at room temperature in a petri plate. Finally, these glass beads were washed thrice with 20 ml milli-Q water followed by 20 ml ethanol, dried in oven at 80° C. for 1 h., and stored at 4° C. (See FIG. 14).

### Example 11

### Conjugation of Glass Beads Bearing Terminal Carboxylic Acid Groups with Anti-EpCAM Antibody, Anti-CD45 Antibody and Transferrin

200 mg glass beads of example 10 were added to a solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. Reaction mixture was decanted and the glass beads isolated were washed with 1 ml phosphate buffer (pH 7.4) thrice, added to 2 ml phosphate buffer (pH 7.4), followed by addition of 10 μg anti-EpCAM antibody. The reaction mixture was kept for 4 h over rocker shaker at room temperature in a petri plate. The glass beads isolated, were washed with 2 ml phosphate buffer (pH 7.4) thrice, and stored in 2 ml PBS (pH 7.4) at 4° C.

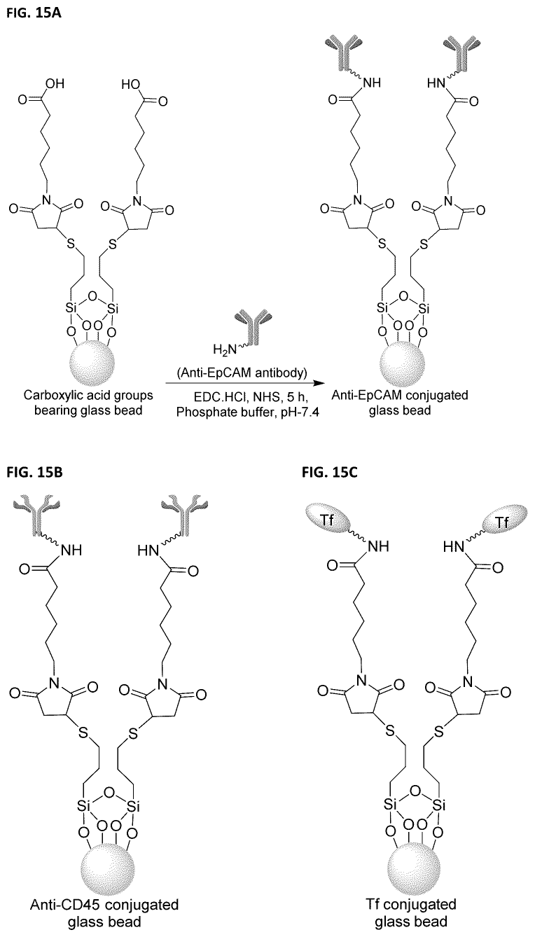

Anti-CD45 antibody and transferrin were similarly conjugated under identical reaction conditions. The structures of these conjugates are shown in FIGS. 15A, 15B, and 15C respectively.

### Example 12

### Conjugation of Mercapto-PEG-Carboxylic Acid (SH-PEG-COOH) Spacer with Glass Beads Bearing Disulfide Linkages

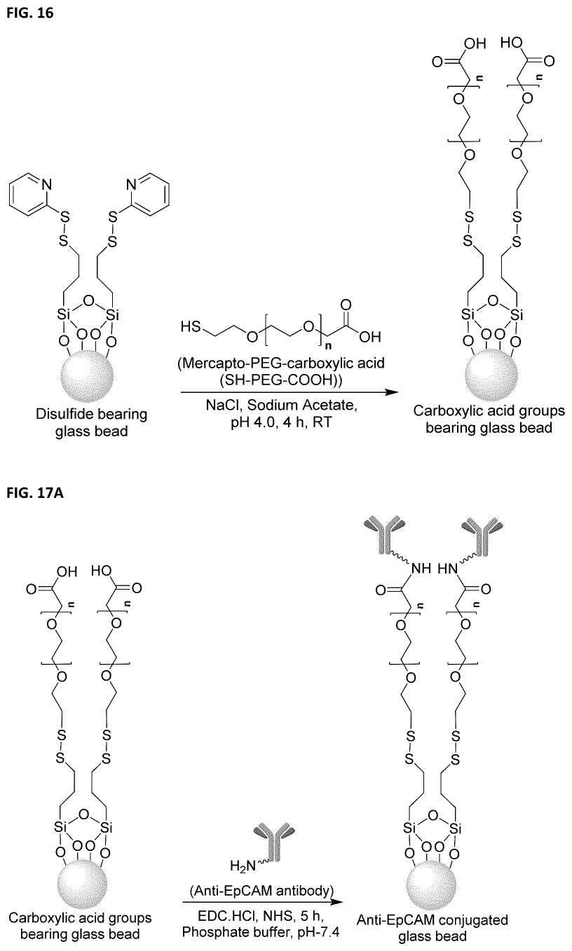

25 mM mercapto-PEG-carboxylic acid solution was prepared in a buffer (pH 4.0) (1 M sodium chloride (NaCl), 0.1 M sodium acetate). 1 g glass beads of example 4 were incubated in above solution for 4 h over rocker shaker at room temperature in a petri plate. These glass beads were washed thrice with 20 ml milli-Q water followed by 20 ml ethanol, dried in oven at 80° C. for 1 h. and stored at 4° C. (See FIG. 16).

### Example 13

### Conjugation of Glass Beads Bearing Terminal Carboxylic Acid Groups with Anti-EpCAM Antibody, Anti-CD45 Antibody and Transferrin

200 mg glass beads of example 12 were added in a solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. Reaction mixture was decanted and glass beads were washed with 1 ml phosphate buffer (pH 7.4) thrice. Glass beads were added to 2 ml phosphate buffer (pH 7.4), followed by addition of 10 μg anti-EpCAM antibody. The reaction mixture was kept for 4 h over rocker shaker at room temperature in a petri plate. These glass beads were washed with 2 ml phosphate buffer (pH 7.4) thrice, and stored in 2 ml PBS (pH 7.4) at 4° C.

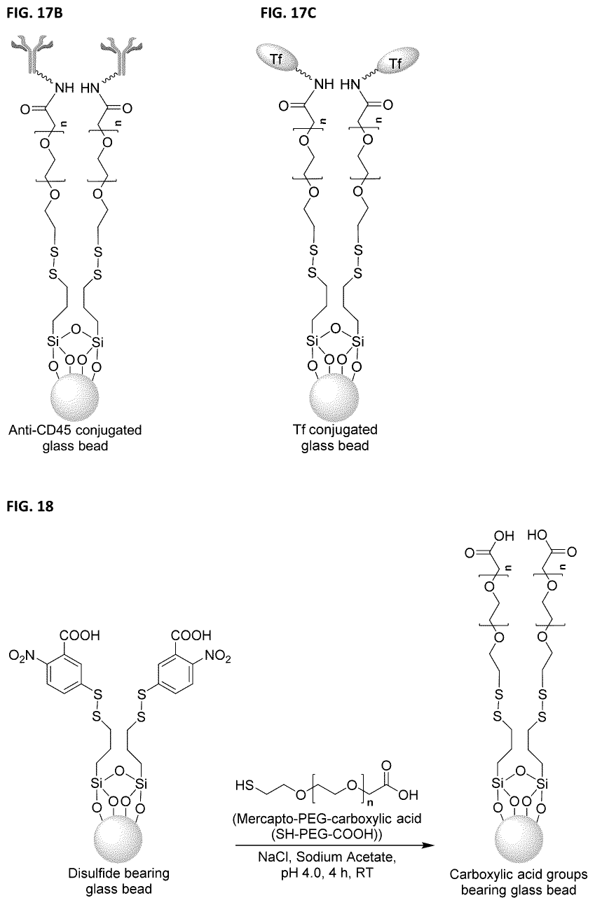

Anti-CD45 antibody and transferrin were similarly conjugated under identical reaction conditions. The structures of these conjugates are shown in FIGS. 17A, 17B, and 17C respectively.

### Example 14

### Synthesis of Glass Beads Bearing Terminal Carboxylic Acid Groups

25 mM SH-PEG-COOH solution was prepared in a pH 4.0 buffer (1 M sodium chloride (NaCl), 0.1 M sodium acetate). 1 g glass beads of example 7 were incubated in 20 ml of the above solution for 4 h over rocker shaker at room temperature in a petri plate. These glass beads were washed thrice with 20 ml milli-Q water followed by 20 ml ethanol, dried in oven at 80° C. for 1 h. and stored at 4° C. (See FIG. 18).

### Example 15

### Conjugation of Glass Beads Bearing Terminal Carboxylic Acid Groups with Anti-EpCAM Antibody, Anti-CD45 Antibody and Transferrin

200 mg glass beads of example 14 were added to a solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. Reaction mixture was decanted and glass beads were washed with 1 ml phosphate buffer (pH 7.4) thrice. These glass beads were added to 2 ml phosphate buffer (pH 7.4), followed by addition of 10 μg of anti-EpCAM antibody. The reaction mixture was kept for 4 h over rocker shaker at room temperature in a petri plate. These glass beads were washed with 2 ml phosphate buffer (pH 7.4) thrice and stored in 2 ml PBS (pH 7.4) at 4° C.

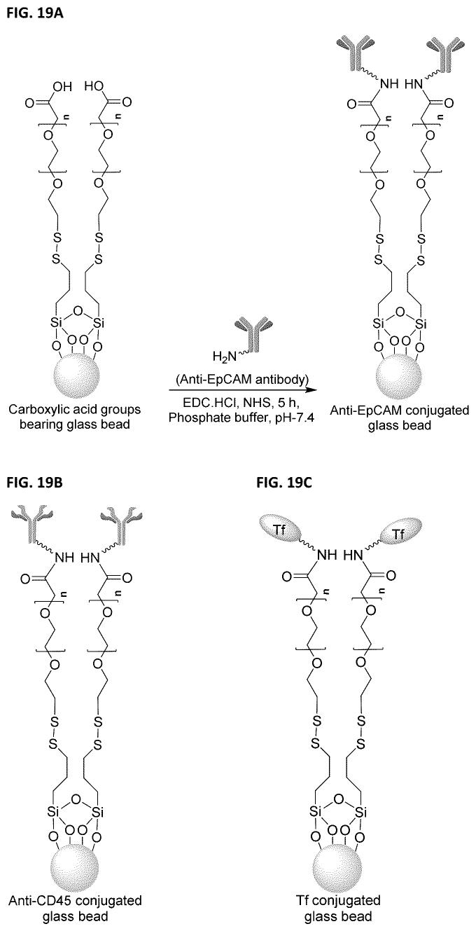

Anti-CD45 antibody and transferrin were similarly conjugated under identical reaction conditions. The structures of these conjugates are shown in FIGS. 19A, 19B, and 19C respectively.

### Example 16

### Conjugation of Hydroxylated Glass Surface with (3-Mercaptopropyl)Trimethoxysilane

Solutions of (3-mercaptopropyl)trimethoxysilane (MPTMS) (1%, 3%, 5%, 10%, 20%, 40%, 60%, 80% and 100%) in water:ethanol (3:2 v/v) were hydrolyzed in 15 ml tubes for 12 h prior to use. 1 g of glass slide bearing hydroxyl functional groups was immersed in 15 ml hydrolyzed solution of MPTMS in a separate petri plate for each concentration and shaken for 12 h over rocker shaker at room temperature Glass slides were then flipped and shaken again for 12 h over rocker shaker at room temperature. Finally, glass slides were washed thrice with 15 ml milli-Q water followed by 15 ml acetone. Glass slides were dried in oven at 80° C. for 1 h. Thiol group density was determined by UV-Vis spectroscopy analysis (See Table 1).

TABLE 1

| Sr. No. | Silane (wt %) | Thiol groups/mm² |
|---|---|---|
| 1 | 1 | 5.05 × 10¹² |
| 2 | 3 | 2.27 × 10¹³ |
| 3 | 5 | 1.30 × 10¹³ |
| 4 | 10 | 1.01 × 10¹³ |
| 5 | 20 | 8.05 × 10¹² |
| 6 | 40 | 1.72 × 10¹³ |
| 7 | 60 | 3.53 × 10¹³ |
| 8 | 80 | 2.42 × 10¹³ |
| 9 | 100 | 4.15 × 10¹³ |

To those skilled in the art, it would be apparent that the reaction can be carried out using different glass substrate geometries selected from glass beads of different diameters, glass capillary tubes of different internal diameters, and glass cover slips.

### Example 17

### Preparation of Iron Oxide Nanoparticles by Co-Precipitation Method

1.1 g iron (II) chloride tetrahydrate (FeCl₂4H₂O) and 4.0 g iron (III) chloride hexahydrate (FeCl₃·6H₂O) were dissolved in 75 ml milli-Q water. The pH of the solution was adjusted to 10 by adding aqueous ammonia solution with continuous stirring. It was heated at 80° C. in water bath for 30 min with continuous agitation and then cooled to room temperature. The reaction flask was placed on a magnet and iron oxide nanoparticles were allowed to settle and the supernatant was decanted. Fresh milli-Q water was added to the flask and sonicated to resuspend the synthesized iron oxide nanoparticles. The reaction flask was placed on magnet and the supernatant was decanted. This procedure was repeated until the supernatant became colourless. Finally, the particles were washed with ethanol, dried in oven at 80° C. and stored at room temperature.

### Example 18

### Functionalization of Iron Oxide Nanoparticles with (3-Mercaptopropyl)Trimethoxysilane

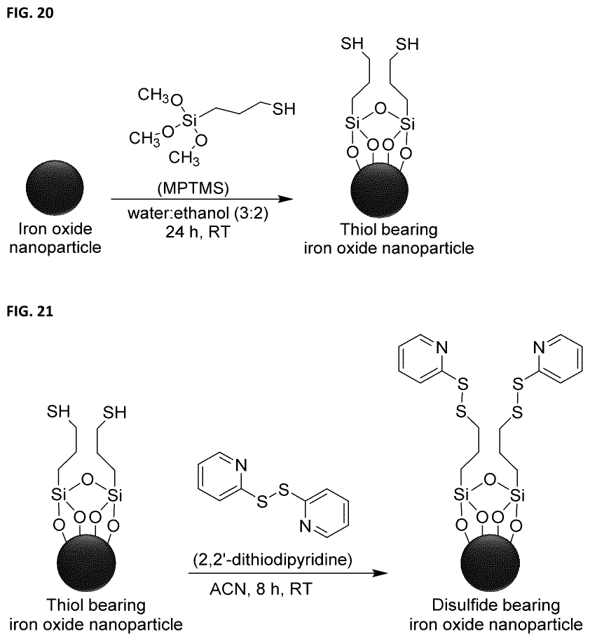

A solution of 5% (v/v) (3-mercaptopropyl)trimethoxysilane (MPTMS) in water:ethanol (3:2 v/v) was hydrolyzed for 12 h prior to use. 500 mg iron oxide nanoparticles of example 17 were dispersed in 5 ml of above solution and reaction mixture was vortexed for 12 h. The reaction product was then separated magnetically and the supernatant was decanted. The particles were then washed with 10 ml milli-Q water three times, washed with ethanol, dried at 80° C. in hot air oven and stored at 4° C. (See FIG. 20).

### Example 19

### Conjugation of 2,2′-Dithiodipyridine (DTDP) Spacer with Thiol Bearing Iron Oxide Nanoparticles

1 mM DTDP solution was prepared in acetonitrile (ACN). 300 mg thiol bearing iron oxide nanoparticles of example 18 were immersed in 2 ml of above solution and reaction mixture was vortexed for 8 h. The reaction product was then separated magnetically and the supernatant was removed. The particles were washed thrice with 2 ml acetonitrile followed by 2 ml acetone, air-dried for 1 h and stored at 4° C. (See FIG. 21).

### Example 20

### Conjugation of 3-Mercaptopropionic Acid (MPA) Spacer with Iron Oxide Nanoparticles Bearing Disulfide Linkages

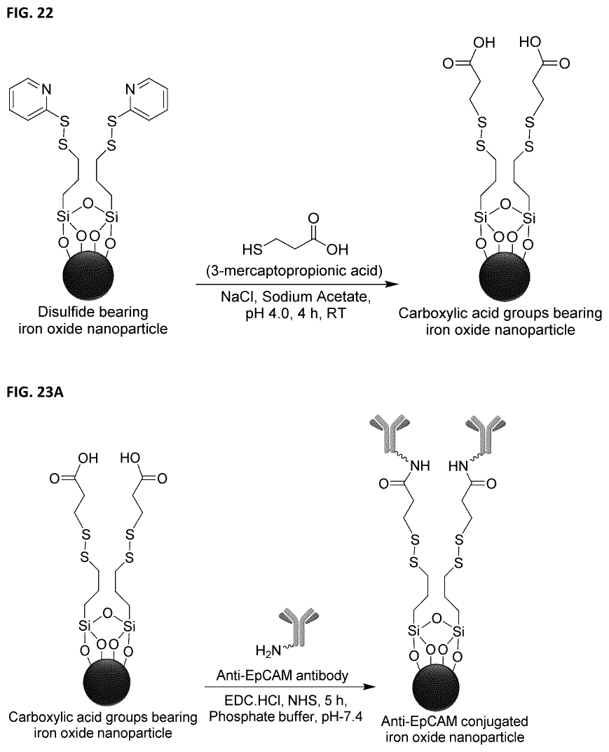

200 mg iron oxide nanoparticles of example 19 were incubated in 2 ml 25 mM MPA solution prepared in a pH 4.0 buffer (1 M sodium chloride (NaCl), 0.1 M sodium acetate) and reaction mixture was vortexed for 4 h. The reaction product was then separated magnetically and the supernatant was decanted. Finally, the particles were washed thrice with 2 ml milli-Q water followed by 2 ml acetone, air-dried for 1 h and stored at 4° C. (See FIG. 22).

### Example 21

### Conjugation of Iron Oxide Nanoparticles Bearing Terminal Carboxylic Acid Groups with Anti-EpCAM Antibody, Anti-CD45 Antibody and Transferrin

10 mg iron oxide nanoparticles of example 20 were dispersed in a solution of N-(3-dimethylaminopropyl)-N-ethylcarbodiimide hydrochloride (4 mg/ml) and N-hydroxy succinimide (4 mg/ml) in 1 ml phosphate buffer (pH 7.4) and incubated for 1 h. They were separated using a magnet and washed with 1 ml phosphate buffer (pH 7.4) thrice, redispersed in 2 ml phosphate buffer (pH 7.4), followed by addition of 10 μg anti-EpCAM antibody. The reaction mixture was vortexed for 4 h. The nanoparticles were separated using a magnet and washed with 1 ml phosphate buffer (pH 7.4) thrice and stored in 200 μl PBS (pH 7.4) at 4° C.

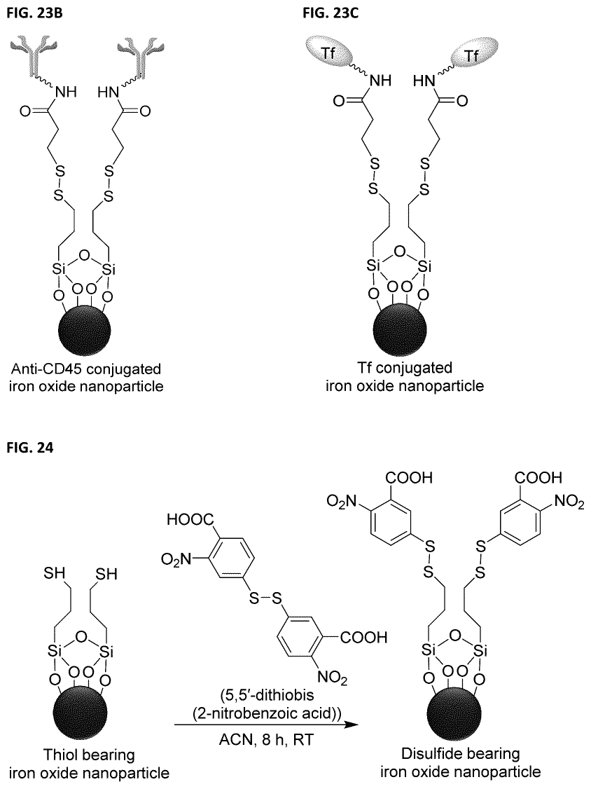

Anti-CD45 antibody and transferrin were similarly conjugated under identical reaction conditions. The structures of these conjugates are shown in FIGS. 23A, 23B, and 23C respectively.

### Example 22

### Conjugation of 5,5′-Dithiobis(2-Nitrobenzoic Acid) (DTNB) Spacer with Thiol Bearing Iron Oxide Nanoparticles

1 mM DTNB solution was prepared in acetonitrile (ACN). 300 mg thiol functionalized iron oxide nanoparticles of example 18 were added to above solution and reaction mixture was vortexed for 8 h. The reaction product was then separated magnetically and the supernatant was decanted. The nanoparticles were washed thrice with 2 ml acetonitrile followed by 2 ml acetone. Iron oxide nanoparticles bearing carboxylic acid groups were air-dried for 1 h and stored at 4° C. (See FIG. 24).

### Example 23

### Conjugation of 3-Mercaptopropionic Acid (MPA) Spacer with Disulfide Bearing Iron Oxide Nanoparticles

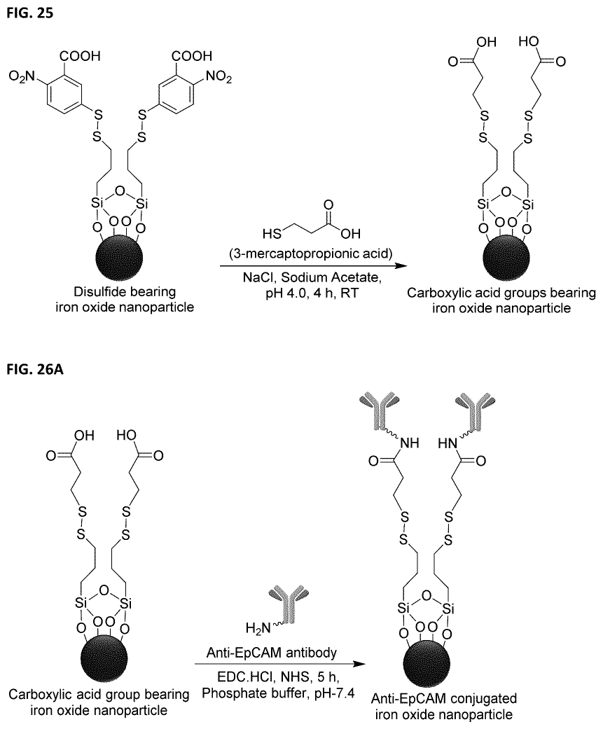

200 mg iron oxide nanoparticles of example 18 were incubated in 2 ml 25 mM MPA solution in a pH 4.0 buffer (1 M sodium chloride (NaCl), 0.1 M sodium acetate) and reaction mixture was vortexed for 4 h. The nanoparticles were then separated magnetically and the supernatant was decanted. The nanoparticles bearing carboxylic acid groups were washed thrice with 2 ml milli-Q water followed by 2 ml acetone, were air-dried for 1 h and stored at 4° C. (See FIG. 25).

### Example 24

### Conjugation of Iron Oxide Nanoparticles Bearing Terminal Carboxylic Acid Groups with Anti-EpCAM Antibody, Anti-CD45 Antibody and Transferrin

10 mg iron oxide nanoparticles of example 23 were dispersed in solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. These nanoparticles were separated using magnet and washed with 1 ml phosphate buffer (pH 7.4) thrice, these nanoparticles were redispersed in 2 ml phosphate buffer (pH 7.4), followed by addition of 10 μg anti-EpCAM antibody. The reaction mixture was vortexed for 4 h, followed by the recovery of nanoparticles using a magnet. The nanoparticles were washed with 1 ml phosphate buffer (pH 7.4) thrice, stored in 200 μl PBS (pH 7.4) at 4° C.

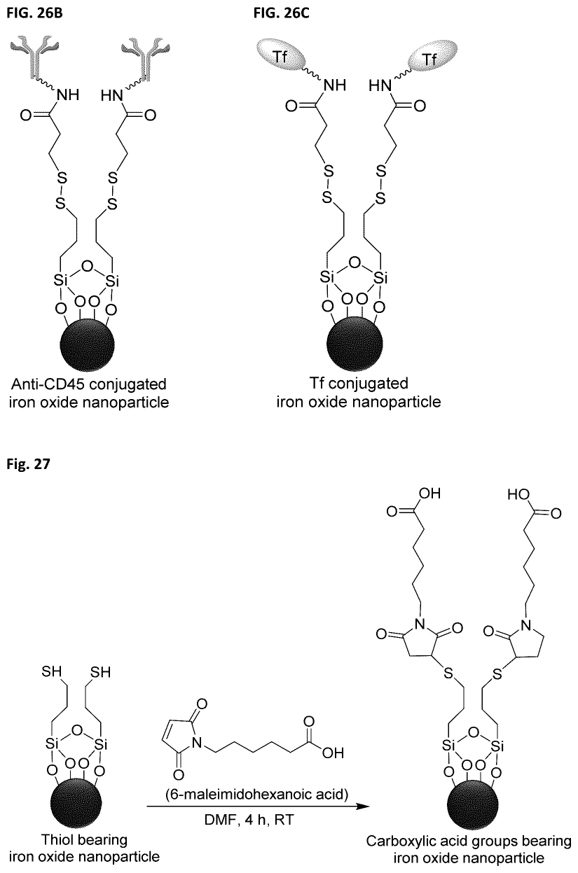

Anti-CD45 antibody and transferrin were similarly conjugated under identical reaction conditions. The structures of these conjugates are shown in FIGS. 26A, 26B, and 26C.

### Example 25

### Conjugation of 6-Maleimidohexanoic Acid Spacer with Thiol Bearing Iron Oxide Nanoparticles

25 mM 6-maleimidohexanoic acid solution was prepared in DMF. 200 mg thiol bearing iron oxide nanoparticles of example 18 were incubated in 2 ml of the above solution and the reaction mixture was vortexed for 4 h. The nanoparticles were then separated magnetically and after decanting the supernatant, washed thrice with 2 ml milli-Q water followed by 2 ml acetone, air-dried for 1 h and stored at 4° C. (See FIG. 27).

### Example 26

### Conjugation of Iron Oxide Nanoparticles Bearing Terminal Carboxylic Acid Groups with Anti-EpCAM Antibody, Anti-CD45 Antibody and Transferrin

10 mg iron oxide nanoparticles of example 25 were dispersed in solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. These nanoparticles were separated using magnet and washed with 1 ml phosphate buffer (pH 7.4) thrice, redispersed in 2 ml phosphate buffer (pH 7.4), followed by addition of 10 μg anti-EpCAM antibody. The reaction mixture was vortexed for 4 h, separated using a magnet and washed with 1 ml phosphate buffer (pH 7.4) thrice, and stored in 200 μl PBS (pH 7.4) at 4° C.

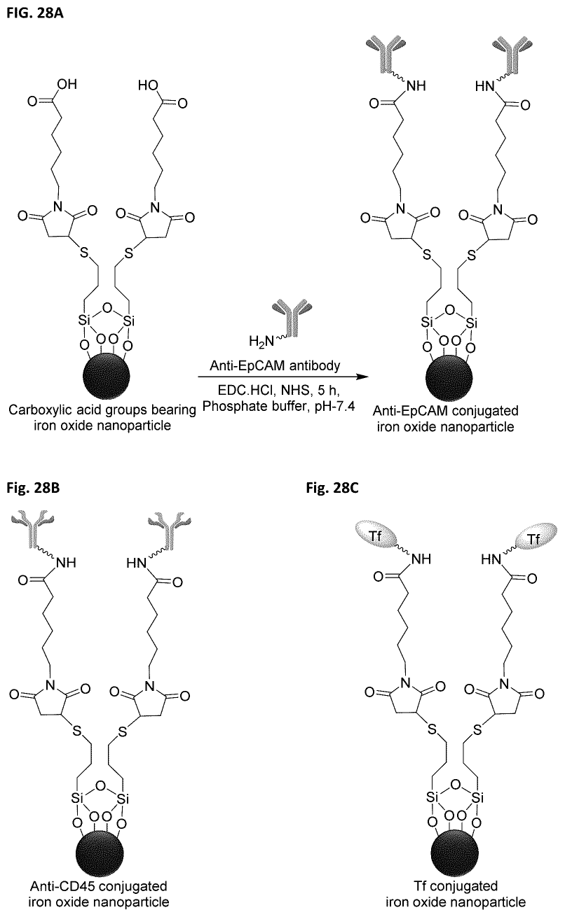

Anti-CD45 antibody and transferrin were similarly conjugated under identical reaction conditions. The structures of these conjugates are shown in FIGS. 28A, 28B, and 28C.

### Example 27

### Conjugation of Graphene Oxide with (3-Mercaptopropyl)Trimethoxysilane

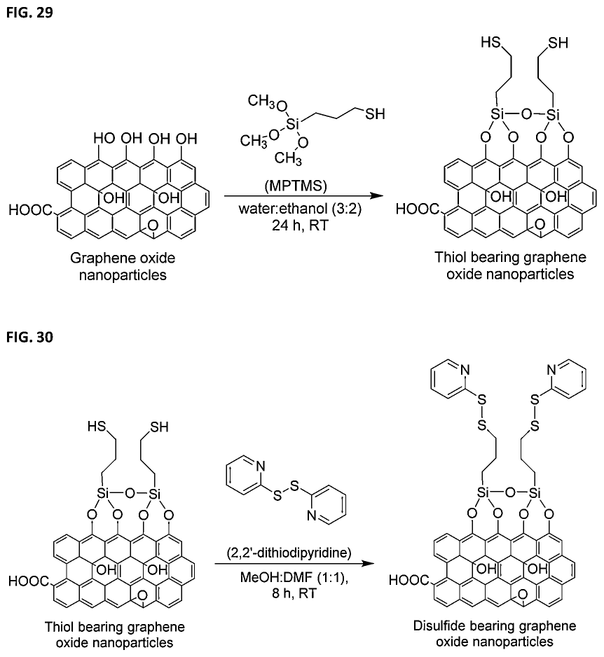

A solution of 5% (v/v) 3-mercaptopropyl)trimethoxysilane (MPTMS) in water:ethanol (3:2 v/v) was hydrolyzed for 12 h prior to use. 500 mg graphene oxide was dispersed in 5 ml of above solution and reaction mixture was vortexed for 12 h. The graphene nanoparticles were filtered, washed with 10 ml milli-Q water three times, then washed with ethanol, dried at 60° C. in hot air oven and stored at 4° C. (See FIG. 29).

### Example 28

### Conjugation of 2,2′-Dithiodipyridine (DTDP) Spacer with Thiol Bearing Graphene Oxide Nanoparticles

1 mM DTDP solution was prepared in MeOH:DMF (1:1 v/v). 300 mg graphene oxide nanoparticles of example 27 were added and the reaction mixture was vortexed for 8 h. The nanoparticles were filtered and washed with 10 ml milli-Q water three times, washed with methanol, dried at 60° C. in hot air oven and stored at 4° C. (See FIG. 30).

### Example 29

### Conjugation of 3-Mercaptopropionic Acid (MPA) Spacer with Graphene Oxide Nanoparticles Bearing Disulfide Linkages

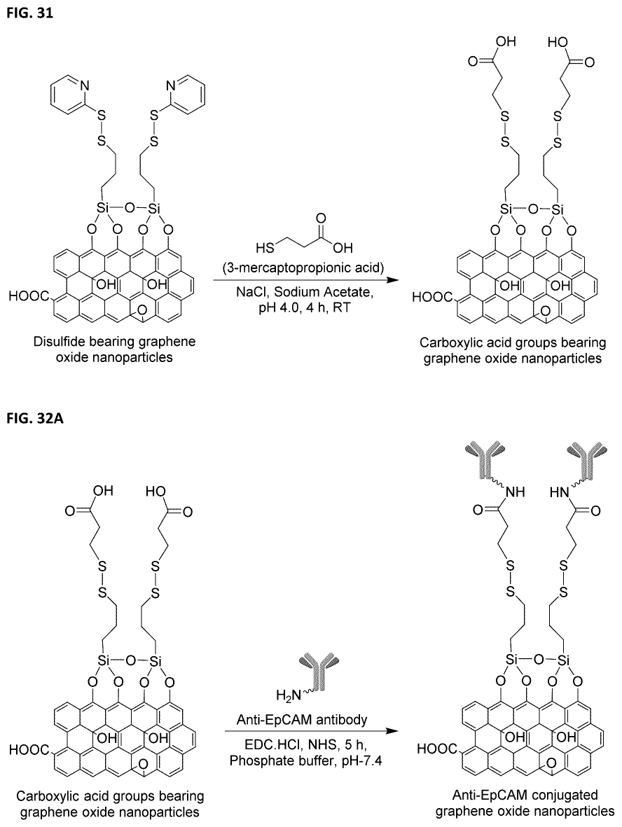

25 mM MPA solution was prepared in a pH 4.0 buffer (1 M sodium chloride (NaCl), 0.1 M sodium acetate). 200 mg graphene oxide nanoparticles of example 28 were added in 2 ml of above solution and the reaction mixture was vortexed for 4 h. The graphene oxide nanoparticles were filtered and washed with 10 ml milli-Q water three times. Followed by ethanol and dried at 60° C. in hot air oven and stored at 4° C. (See FIG. 31).

### Example 30

### Conjugation of Graphene Oxide Nanoparticles Bearing Terminal Carboxylic Acid Groups with Anti-EpCAM Antibody, Anti-CD45 Antibody and Transferrin

10 mg graphene oxide nanoparticles of example 29 were dispersed in solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. These graphene oxide nanoparticles were filtered and washed with 1 ml phosphate buffer (pH 7.4) thrice, redispersed in 2 ml phosphate buffer (pH 7.4), followed by addition of 10 μg anti-EpCAM antibody. The reaction mixture was vortexed for 4 h, followed by filtration and washing with 1 ml phosphate buffer (pH 7.4) thrice and stored in 200 μl PBS (pH 7.4) at 4° C.

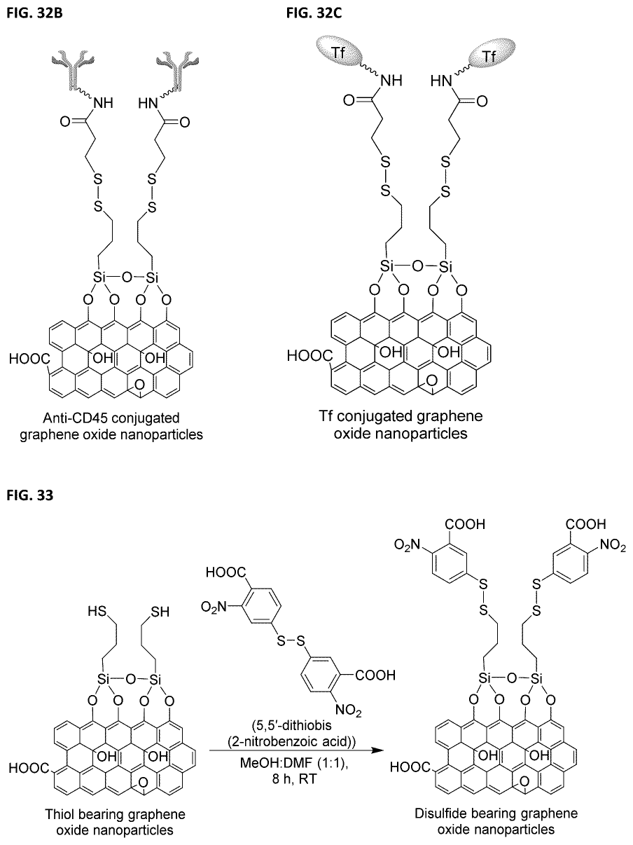

Anti-CD45 antibody and transferrin were similarly conjugated under identical reaction conditions. The structures of these conjugates are shown in FIGS. 32A, 32B, and 32C.

### Example 31

### Conjugation of 5,5′-Dithiobis (2-Nitrobenzoic Acid) (DTNB) Spacer with Thiol Bearing Graphene Oxide Nanoparticles

1 mM DTNB solution was prepared in MeOH:DMF (1:1 v/v). 300 mg graphene oxide of example 27 was added to it and the reaction mixture was vortexed for 8 h. The nanoparticles were filtered and washed with 10 ml milli-Q water three times, washed with methanol, dried at 60° C. in hot air oven and stored at 4° C. (See FIG. 33).

### Example 32

### Conjugation of 3-Mercaptopropionic Acid (MPA) Spacer with Graphene Oxide Nanoparticles Bearing Disulfide Linkages

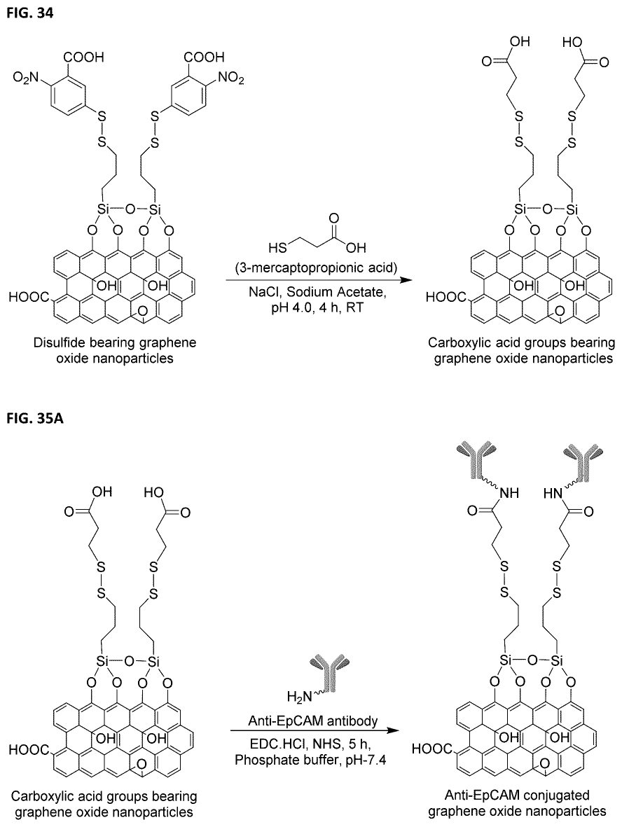

25 mM 3-mercaptopropionic acid (MPA) solution was prepared in a pH 4.0 buffer (1 M sodium chloride (NaCl), 0.1 M sodium acetate). 200 mg graphene oxide nanoparticles of example 31 were added in 2 ml of above solution. The reaction mixture was vortexed for 4 h. and filtered. These nanoparticles were washed with 10 ml milli-Q water three times, washed with ethanol and dried at 60° C. in hot air oven and stored at 4° C. (See FIG. 34).

### Example 33

### Conjugation of Graphene Oxide Nanoparticles Bearing Terminal Carboxylic Acid Groups with Anti-EpCAM Antibody, Anti-CD45 Antibody and Transferrin

10 mg graphene oxide nanoparticles of example 32 were dispersed in solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. These nanoparticles were filtered and washed with 1 ml phosphate buffer (pH 7.4) thrice, redispersed in 2 ml phosphate buffer (pH 7.4), followed by addition of 10 μg anti-EpCAM antibody. The reaction mixture was vortexed for 4 h, followed by filtration. The nanoparticles were washed with 1 ml phosphate buffer (pH 7.4) thrice and stored in 200 μl PBS pH 7.4 at 4° C.

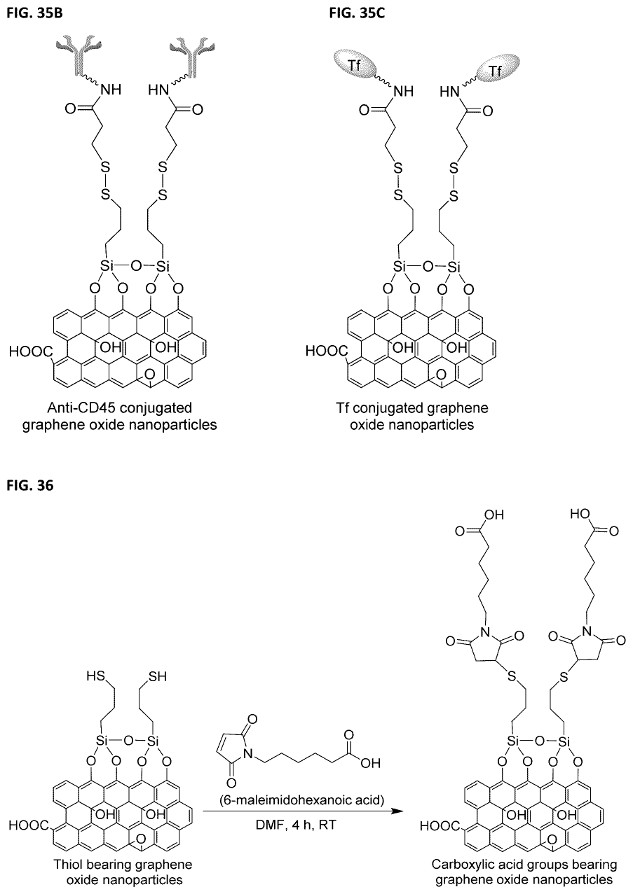

Anti-CD45 antibody and transferrin were similarly conjugated under identical reaction conditions. The structures of these conjugates are shown in FIGS. 35A, 35B, and 35C.

### Example 34

### Conjugation of 6-Maleimidohexanoic Acid Spacer with Thiol Bearing Graphene Oxide Nanoparticles

25 mM 6-maleimidohexanoic acid solution was prepared in DMF. 200 mg graphene oxide nanoparticles of example 27 were added in 2 ml of above solution and reaction mixture was vortexed for 4 h. The nanoparticles were filtered, washed with 10 ml milli-Q water three times, then with ethanol, dried at 60° C. in hot air and stored at 4° C. (See FIG. 36).

### Example 35

### Conjugation of Graphene Oxide Nanoparticles Bearing Terminal Carboxylic Acid Groups with Anti-EpCAM Antibody, Anti-CD45 Antibody and Transferrin

10 mg graphene oxide nanoparticles of example 34 were dispersed in solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. These nanoparticles were filtered and washed with 1 ml phosphate buffer (pH 7.4) thrice, and were redispersed in 2 ml phosphate buffer (pH 7.4), followed by addition of 10 μg anti-EpCAM antibody. The reaction mixture was vortexed for 4 h, followed by filtration and washing with 1 ml phosphate buffer (pH 7.4) thrice and stored in 200 IA PBS (pH 7.4) at 4° C.

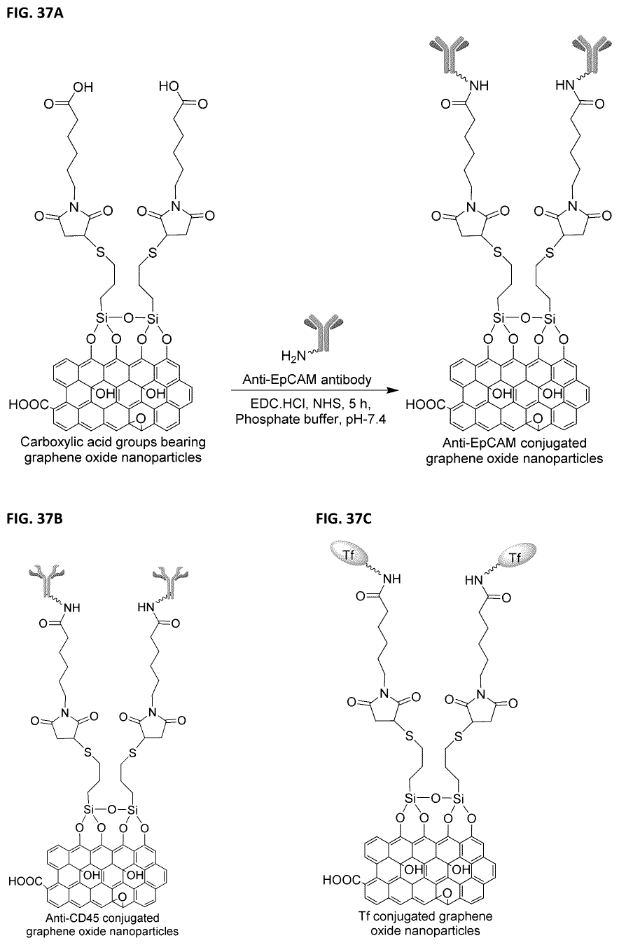

Anti-CD45 antibody and transferrin were similarly conjugated under identical reaction conditions. The structures of these conjugates are shown in FIGS. 37A, 37B, and 37C.

To those skilled in the art, it would be apparent that the reaction can be carried out using different geometries of carbon allotropes.

### Example 36

### Conjugation of Glass Beads Using Two Different Silanes

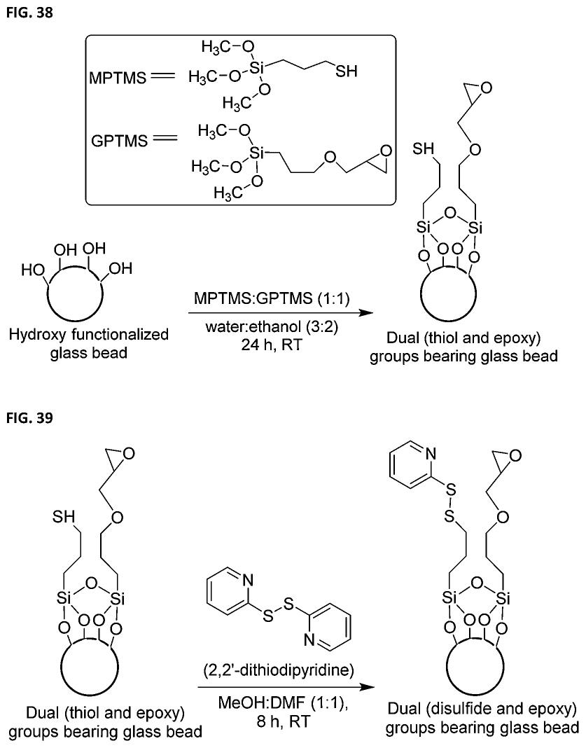

A solution of 5% (3-mercaptopropyl)trimethoxysilane (MPTMS):(3-Glycidyloxypropyl) trimethoxysilane (GPTMS) (1:1 v/v) in water:ethanol (3:2 v/v) was hydrolyzed for 12 h prior to use. 1 g glass beads of example 2 were immersed in 10 ml of above solution in a petri plate and shaken for 12 h over rocker shaker at room temperature. These glass beads were washed thrice with 10 ml milli-Q water followed by 10 ml acetone, dried in oven at 80° C. for 1 h and stored at 4° C. (See FIG. 38).

### Example 37

### Conjugation of 2,2′-Dithiodipyridine (DTDP) Spacer with Thiol Group of Glass Beads Bearing Two Different Silanes

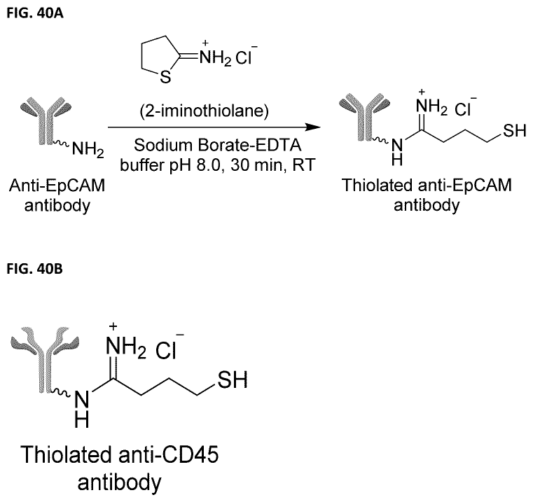

1 mM DTDP solution was prepared in MeOH:DMF (1:1 v/v). 500 mg glass beads of example 36 were immersed in 5 ml of the above solution in a petri plate and shaken for 8 h over rocker shaker at room temperature. The glass beads were washed thrice with 5 ml methanol, and dried at 80° C. in hot air oven for 1 h. and stored at 4° C. (See FIG. 39).

### Example 38

### Thiolation of Anti-EpCAM and Anti-CD45 Antibody with 2-Iminothiolane

10 μg anti-EpCAM antibody was added in 1 ml borate buffer (pH 8.0) (0.1 M sodium borate, 5 mM EDTA, 0.15 M NaCl), followed by addition of 2-iminothiolane-HCl (1 mg/ml) solution. The reaction mixture was vortexed for 30 min at room temperature. The thiolated anti-EpCAM antibody was purified using dialysis membrane (100 kDa) and washed with 1 ml phosphate-EDTA buffer (pH 7.4) (20 mM sodium phosphate, 0.15-M NaCl, 1 mM EDTA) three times and stored at 4° C. in PBS (pH 7.4) (See FIG. 40A). Anti-CD45 antibody was similarly prepared (See FIG. 40B).

### Example 39

### Synthesis of Adsorbent Bearing Anti-EpCAM Antibody Conjugated Through Cleavable Linkage and Anti-CD45 Antibody Conjugated Through Non-Cleavable Linkage

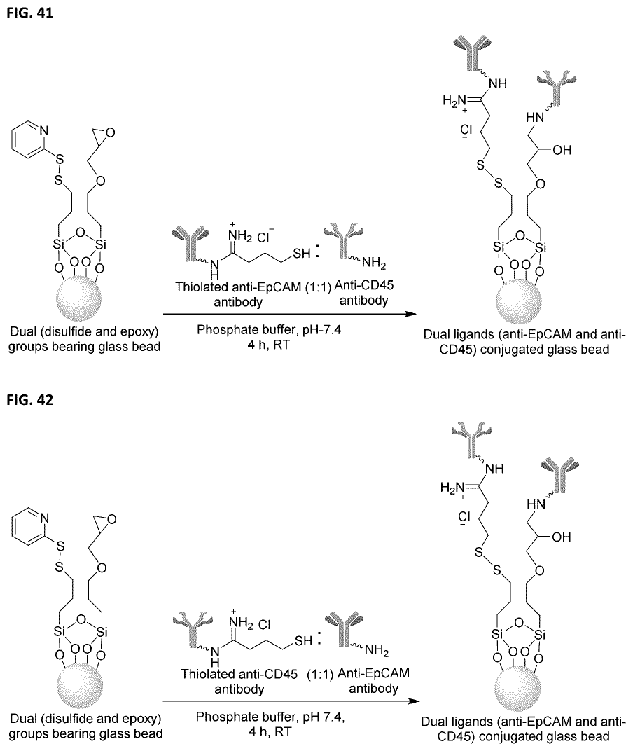

200 mg glass beads of example 37 were added in a solution of 2 ml phosphate buffer (pH 7.4), followed by addition of 5 μg thiolated anti-EpCAM antibody of example 38 and 5 μg anti-CD45 antibody (1:1). The reaction mixture was kept for 4 h over rocker shaker at room temperature in a petri plate. These glass beads were washed with 2 ml phosphate buffer (pH 7.4) thrice and stored in 2 ml PBS (pH 7.4) at 4° C. (See FIG. 41).

### Example 40

### Synthesis of Adsorbent Bearing Anti-EpCAM Antibody Conjugated Through Non-Cleavable Linkage and Anti-CD45 Antibody Conjugated Through Cleavable Linkage

200 mg glass beads of example 37 were added to 2 ml phosphate buffer (pH 7.4), followed by addition of 5 μg anti-EpCAM antibody and 5 μg thiolated anti-CD45 antibody of example 38 (1:1). The reaction mixture was kept for 4 h over rocker shaker at room temperature in a petri plate. Glass beads were washed with 2 ml phosphate buffer (pH 7.4) thrice and stored in 2 ml PBS (pH 7.4) at 4° C. (See FIG. 42).

### Example 41

### Conjugation of N-Succinimidyl-3-(2-Pyridyldithio) Propionate (SPDP) Spacer with Anti-EpCAM and Anti-CD45 Antibody

20 mM solution of SPDP reagent was prepared in DMSO. 10 μg anti-EpCAM antibody was dissolved in 1 ml phosphate-EDTA buffer (20 mM sodium phosphate, 0.15 M NaCl, 1 mM EDTA, pH 7.4) followed by addition of 25 μl SPDP solution. The reaction mixture was vortexed for 30 min at room temperature. The reaction product was then purified using dialysis membrane (100 kDa) and washed with 1 ml phosphate buffer (pH 7.4) three times and stored at 4° C. in PBS (pH 7.4) (See FIG. 43A). Anti-CD45 antibody-SPDP conjugate was synthesized similarly (See FIG. 43B).

### Example 42

### Synthesis of Adsorbent Bearing Anti-EpCAM Antibody Conjugated Through Cleavable Linkage and Anti-CD45 Antibody Conjugated Through Non-Cleavable Linkage

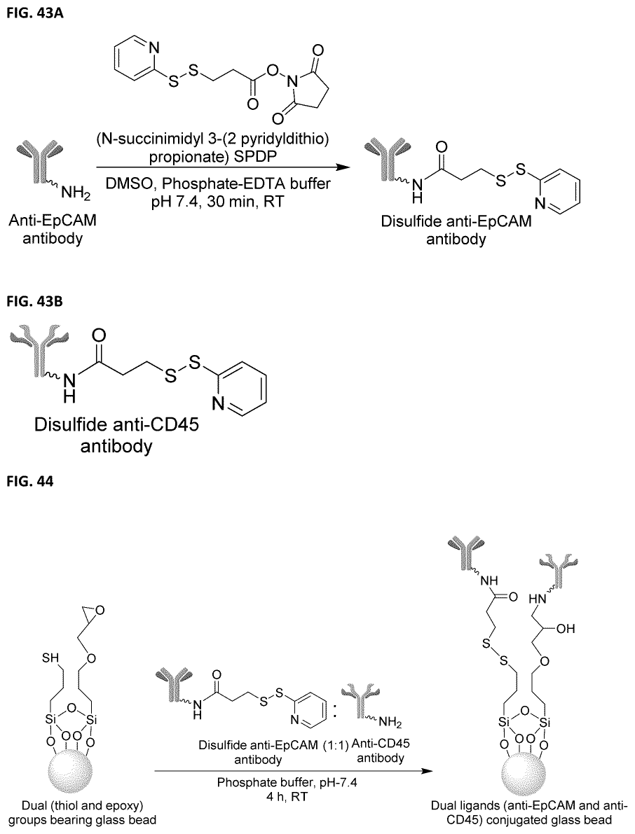

200 mg glass beads of example 36 were added in a 2 ml phosphate buffer (pH 7.4) followed by addition of 5 μg disulphide conjugated anti-EpCAM antibody of example 41 and 5 μg anti-CD45 antibody (1:1). The reaction mixture was kept for 4 h over rocker shaker at room temperature in a petri plate. Glass beads were washed with 2 ml phosphate buffer (pH 7.4) thrice and stored in 2 ml PBS (pH 7.4) at 4° C. (See FIG. 44).

### Example 43

### Synthesis of Adsorbent Bearing Conjugated Anti-EpCAM Antibody Through Non-Cleavable Linkage and Conjugated Anti-CD45 Antibody Through Cleavable Linkage

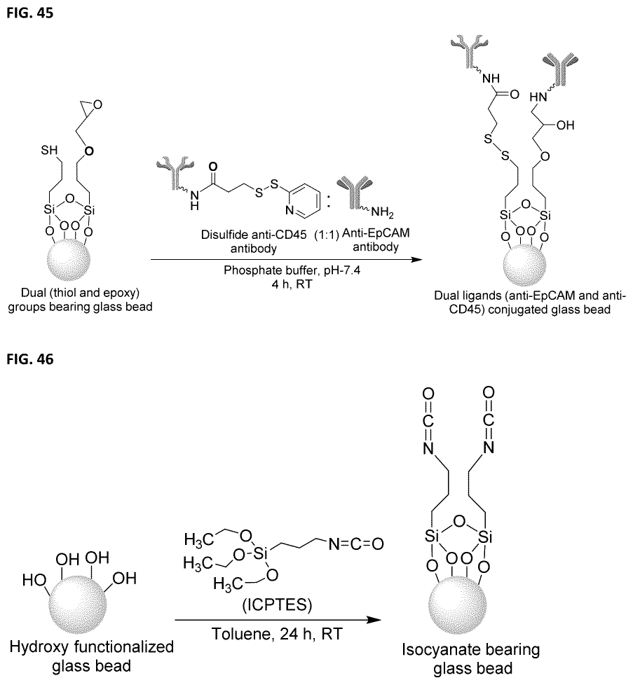

200 mg glass beads of example 36 were added to 2 ml phosphate buffer (pH 7.4) containing 5 μg anti-EpCAM antibody and 5 μg disulfide conjugated anti-CD45 antibody (1:1). The reaction mixture was kept for 4 h over rocker shaker at room temperature in a petri plate. Glass beads were washed with 2 ml phosphate buffer (pH 7.4) thrice and stored in 2 ml PBS (pH 7.4) at 4° C. (See FIG. 45).

### Example 44

### Conjugation of Hydroxylated Glass Beads with (3-Isocyanatopropyl)Triethoxysilane (ICPTES)

5% (3-isocyanatopropyl)triethoxysilane (ICPTES) solution was prepared in toluene. 1 g glass beads of example 2 were added in above solution in a petri plate and shaken for 24 h over rocker shaker at room temperature. These glass beads were then washed thrice with 50 ml toluene followed by 50 ml acetone, dried in oven at 80° C. for 1 h and stored at 4° C. (See FIG. 46).

### Example 45

### Conjugation of Isocyanate Bearing Glass Beads with Anti-EpCAM Antibody, Anti-CD45 Antibody and Transferrin

200 mg glass beads of example 44 were added to 2 ml phosphate buffer. 10 μg anti-EpCAM antibody was added to it. The reaction mixture was kept for 4 h on magnetic stirrer at 5° C. in a vial. These Glass beads were washed with 2 ml phosphate buffer (pH 7.4) thrice and stored in 2 ml PBS pH 7.4 at 4° C.

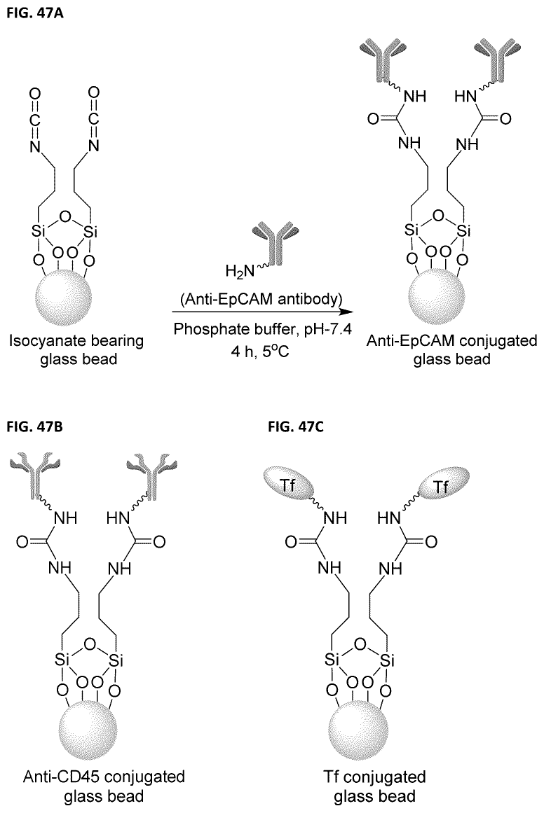

Anti-CD45 antibody and transferrin were similarly conjugated under identical reaction conditions. The structures of these conjugates are shown in FIGS. 47A, 47B, and 47C.

### Example 46

### Conjugation of Glass Beads with (3-Thiocyanatopropyl)Triethoxysilane(TCPTES)

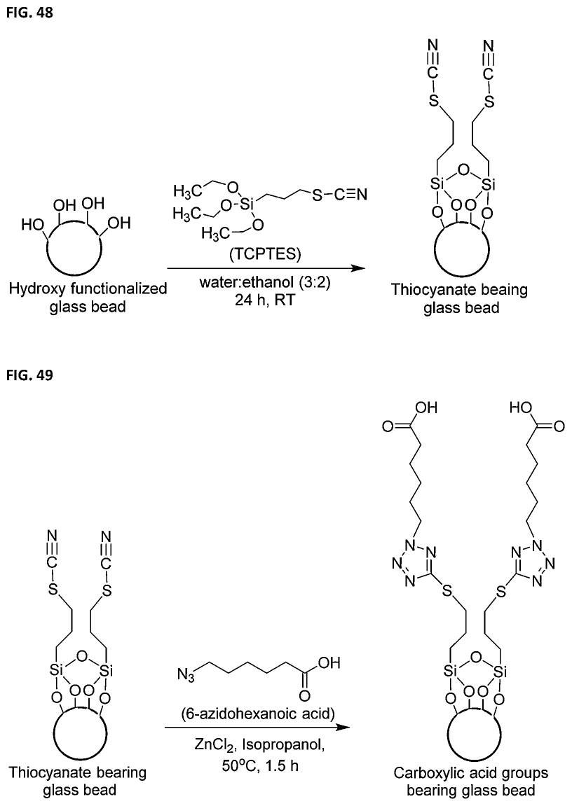

A solution of 5% (3-thiocyanatopropyl)triethoxysilane (TCPTES) in water:ethanol (3:2 v/v) was hydrolyzed for 12 h prior to use. 1 g glass beads of example 2 were added in 10 ml of above solution in a petri plate and shaken for 12 h over rocker shaker at room temperature. The glass beads were then washed thrice with 50 ml milli-Q water followed by 50 ml acetone and dried in oven at 80° C. for 1 h. and stored at 4° C. (See FIG. 48).

### Example 47

### Synthesis of 6-Azidohexanoic Acid

A solution of 6-bromohexanoic acid (2.0 g) in 20 ml dimethyl sulfoxide (DMSO) was heated to 40° C. and sodium azide (NaN₃) (3.3 g) was added in a dropwise manner. The reaction mixture was stirred for 12 h at 80° C. The temperature was brought down to 40° C. and concentrated HCl (3 ml) was added dropwise to this reaction mixture and stirring continued for 12 h. The product was extracted with diethyl ether (5×20 ml). The ether layers were collected, dried over sodium sulphate (Na₂SO₄), filtered, and the solvent was evaporated. The yellow coloured oily product was stored at 4° C.

### Example 48

### Synthesis of Adsorbent Bearing Terminal Carboxylic Acid Groups

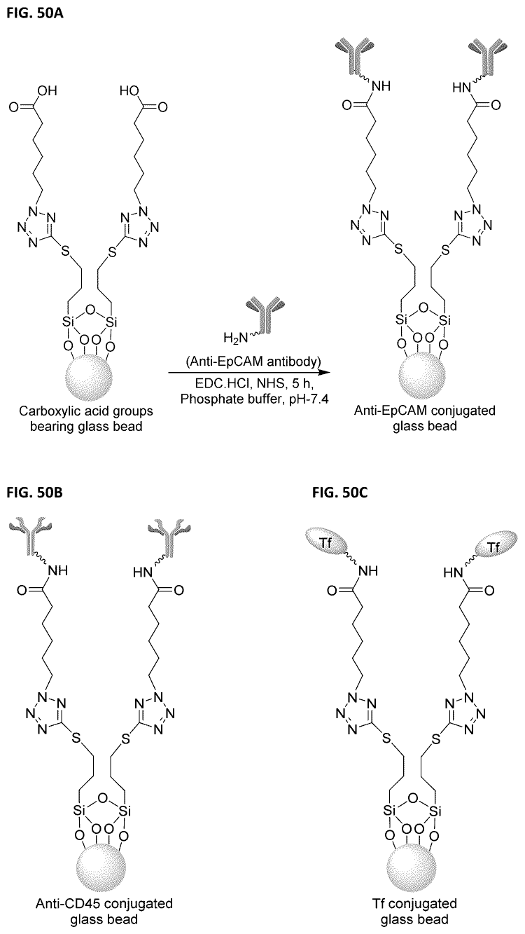

6-azidohexanoic acid (100 mg) and zinc chloride (ZnCl₂) (100 mg) were added to a solution containing 500 mg glass beads of example 47 in isopropanol (5 ml). The reaction mixture was vigorously stirred at 50° C. for 1.5 h. These glass beads were then washed thrice with 10 ml 9% (v/v) HCl followed by 10 ml acetone, and air dried for 1 h. and stored at 4° C. (See FIG. 49).

### Example 49

### Conjugation of Adsorbent Bearing Terminal Carboxylic Acid Groups with Anti-EpCAM Antibody, Anti-CD45 Antibody and Transferrin

200 mg glass beads of example 48 were added to a solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. Reaction mixture was decanted and glass beads washed with 1 ml phosphate buffer at pH 7.4 thrice and these beads were added to 2 ml phosphate buffer (pH 7.4) containing 10 μg anti-EpCAM antibody. The reaction mixture was kept for 4 h over rocker shaker at room temperature in a petri plate. These glass beads were washed with 2 ml phosphate buffer (pH 7.4) thrice and stored in 2 ml PBS (pH 7.4) at 4° C.

Anti-CD45 antibody and transferrin were similarly conjugated under identical reaction conditions. The structures of these conjugates are shown in FIGS. 50A, 50B, and 50C.

### Example 50

### Conjugation of SPDP-PEG₃₆-NHS Ester Spacer with Anti-EpCAM and Anti-CD45 Antibody

20 mM solution of SPDP-PEG₃₆-NHS ester reagent was prepared in DMSO. 10 μg anti-EpCAM antibody was added in 1 ml phosphate-EDTA buffer (20 mM sodium phosphate, 0.15 M NaCl, 1 mM EDTA, pH 7.4) followed by addition of 25 μl SPDP solution. The reaction mixture was vortexed for 30 min at room temperature. The conjugated anti-EpCAM antibody was purified using dialysis membrane (100 kDa), washed with 1 ml phosphate buffer (pH 7.4) three times and stored at 4° C. in PBS (pH 7.4) (See FIG. 51A). Conjugated anti-CD45 antibody was similarly synthesized (See FIG. 51B).

### Example 51

### Synthesis of Adsorbent Bearing Anti-EpCAM Antibody Conjugated Through Cleavable Linkage and Anti-CD45 Antibody Conjugated Through Non-Cleavable Linkage

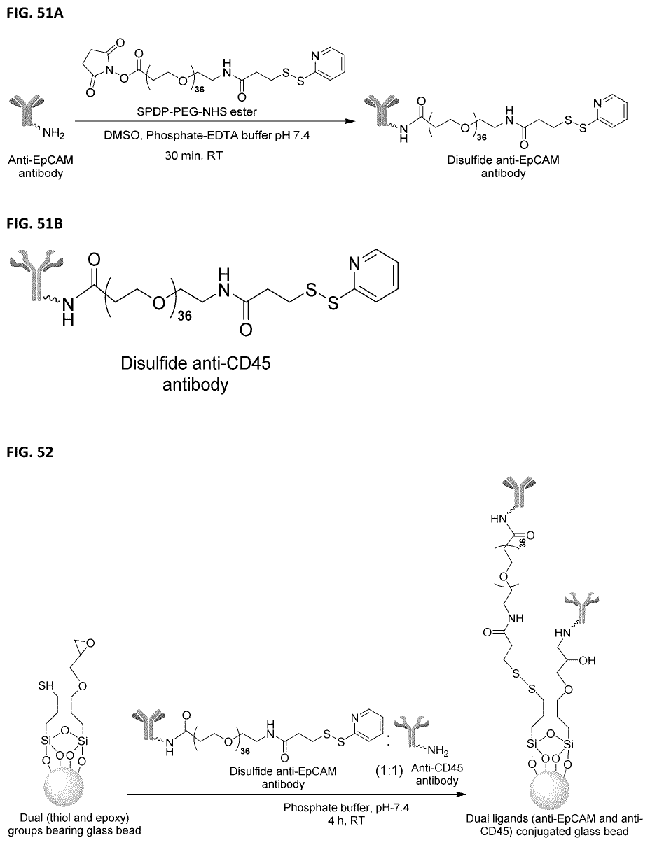

200 mg glass beads of example 36 were added in a solution of 2 ml phosphate buffer (pH 7.4) followed by addition of 5 μg of disulfide anti-EpCAM antibody of example 50 and 5 μg of anti-CD45 antibody (1:1 weight by weight). The reaction mixture was kept for 4 h over rocker shaker at room temperature in a petri plate. These glass beads were washed with 2 ml phosphate buffer (pH 7.4) thrice and stored in 2 ml PBS (pH 7.4) at 4° C. (See FIG. 52).

### Example 52

### Synthesis of Adsorbent Bearing Anti-EpCAM Antibody Conjugated Through Non-Cleavable Linkage and Anti-CD45 Antibody Conjugated Through Cleavable Linkage

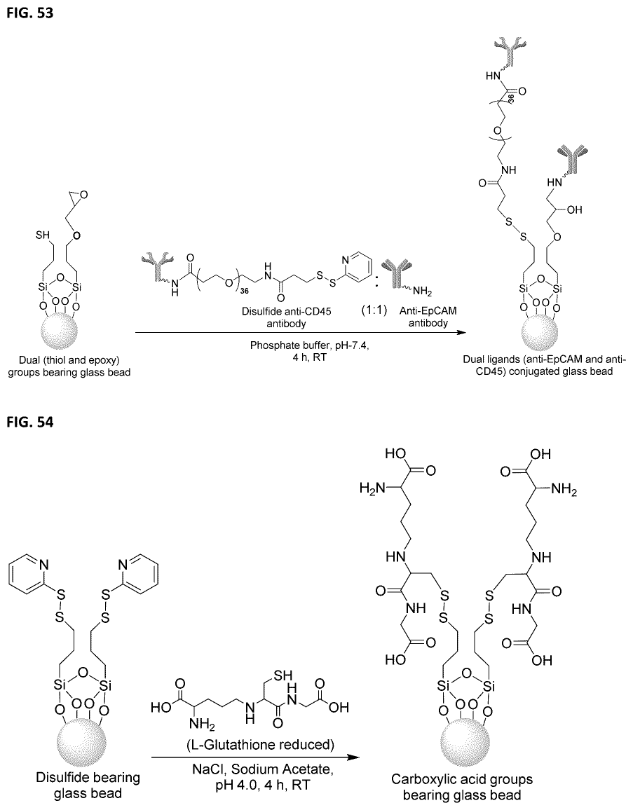

200 mg glass beads of example 36 were added to 2 ml phosphate buffer (pH 7.4) containing 5 μg anti-EpCAM antibody and 5 μg disulfide anti-CD45 antibody of example 50 (1:1 weight by weight). The reaction mixture was kept for 4 h over rocker shaker at room temperature in a petri plate. Glass beads were washed with 2 ml phosphate buffer (pH 7.4) thrice and stored in 2 ml PBS (pH 7.4) at 4° C. (See FIG. 53).

### Example 53

### Conjugation of L-Glutathione Spacer with Glass Beads Bearing Disulfide Linkages

25 mM reduced L-glutathione (GSH) was prepared in a pH 4.0 buffer solution (1 M sodium chloride (NaCl), 0.1 M sodium acetate). 1 g glass beads of example 4 were incubated in 20 ml of the above solution for 4 h over rocker shaker at room temperature in a petri plate. These glass beads were washed thrice with 20 ml milli-Q water, and then with 20 ml ethanol, dried in oven at 80° C. for 1 h. and stored at 4° C. (See FIG. 54).

### Example 54

### Conjugation of Adsorbent Bearing Terminal Carboxylic Acid Groups with Marker Cell Binding Ligands

200 mg glass beads of example 53 were added in a solution of N-(3-dimethylaminopropyl)-N′-ethylcarbodiimide hydrochloride (4 mg/ml) and N-hydroxysuccinimide (4 mg/ml) in 1 ml phosphate buffer (pH 7.4) and incubated for 1 h. Reaction mixture was decanted. Glass beads isolated were washed with 1 ml phosphate buffer (pH 7.4) thrice, and added to 2 ml phosphate buffer (pH 7.4), followed by addition of 10 μg anti-EpCAM antibody. The reaction mixture was kept for 4 h over rocker shaker at room temperature in a petri plate. These glass beads were washed with 2 ml phosphate buffer (pH 7.4) thrice and stored in 2 ml PBS (pH 7.4) at 4° C.

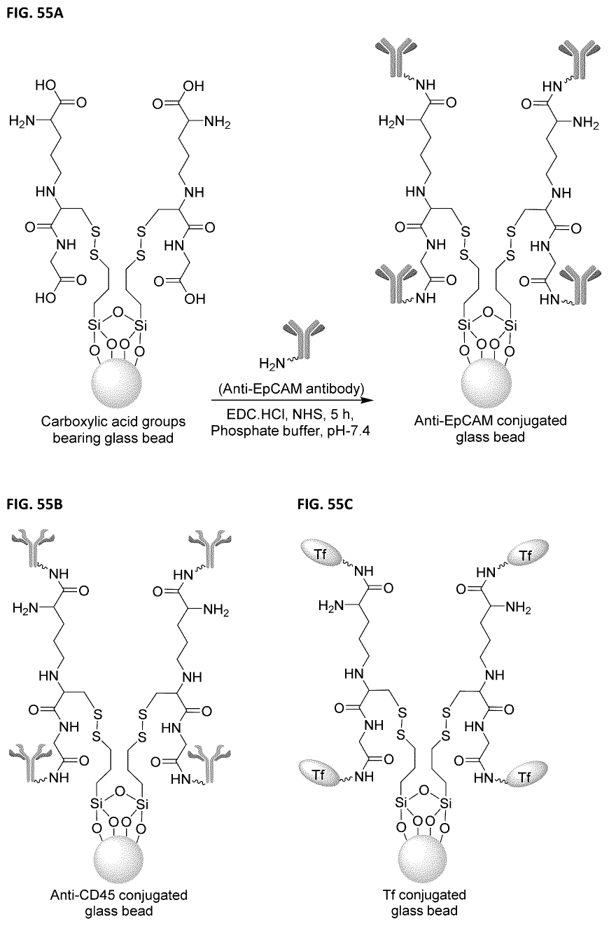

Anti-CD45 antibody and transferrin were similarly conjugated under identical reaction conditions. The structures of these conjugates are shown in FIGS. 55A, 55B, and 55C respectively.

### Example 55

### Dithiothreitol (DTT) Catalysed Cleavage of Disulfide Linkage on Glass Beads

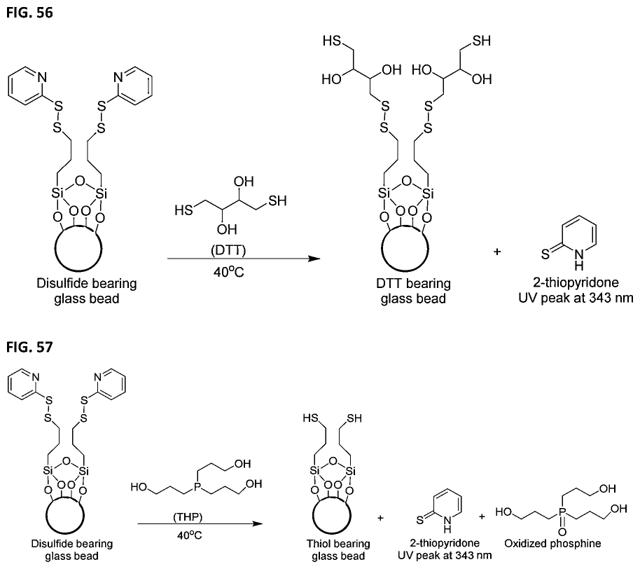

250 mg glass beads of example 4 were treated with 50 mM DTT in different buffer solutions viz. sodium bicarbonate buffer (pH 8.0), tris buffered saline (TBS) (pH6.8 and 8.0), and phosphate-EDTA buffer (pH 7.4) at 40° C. for 30 min. The cleavage of disulfide linkages was then monitored using UV-Vis spectroscopy (See Table 2 and FIG. 56).

TABLE 2

| Sr. no. | Buffer Solutions | Thiol groups/mm² |
|---|---|---|
| 1 | NaHCO₃, pH 8.0 | 3.50 × 10¹⁴ |
| 2 | TBS, pH 8.0 | 1.66 × 10¹⁴ |
| 3 | TBS, pH 6.8 | 1.84 × 10¹⁴ |
| 4 | Phosphate-EDTA buffer, pH 7.4 | 1.81 × 10¹⁴ |

### Example 56

### Time-Dependent DTT Catalysed Cleavage of Disulfide Linkages

250 mg glass beads of example 4 were treated with 100 mM DTT in TBS (pH 8.0) at 40° C. for 30 min. The cleavage of disulfide linkage was then monitored using UV-Vis spectroscopy (See Table 3).

TABLE 3

| Sr. no. | Time (min) | Thiol groups/mm² |
|---|---|---|
| 1 | 5 | 2.90 × 10¹⁴ |
| 2 | 10 | 5.02 × 10¹⁴ |
| 3 | 20 | 6.60 × 10¹⁴ |
| 4 | 30 | 1.60 × 10¹⁵ |

### Example 57

### Time-Dependent Tris(Hydroxypropyl)Phosphine (THP) Catalysed Cleavage of Disulfide Linkages

250 mg glass beads of example 4 were treated with 200 mM THP in TBS (pH 8.0) at 40° C. for 30 min. The cleavage of disulfide linkage was monitored using UV-Vis spectroscopy (See Table 4 and FIG. 57).

TABLE 4

| Sr. no. | Time (min) | Thiol groups/mm² |
|---|---|---|
| 1 | 5 | 1.86 × 10¹⁴ |
| 2 | 10 | 3.17 × 10¹⁴ |
| 3 | 20 | 3.65 × 10¹⁴ |
| 4 | 30 | 9.20 × 10¹⁴ |

### Example 58

### Concentration Dependent DTT and THP Catalysed Cleavage of Disulfide Linkages

250 mg glass beads of example 4 were treated with DTT (50 mM and 100 mM) and THP (100 mM and 200 mM) in TBS (pH 8.0) at 40° C. for 30 min. The cleavage of disulfide linkage was monitored using UV-Vis spectroscopy (See Table 5 and FIGS. 56, 57).

TABLE 5

| Sr. no. | Reagent Concentration | Thiol groups/mm² |
|---|---|---|
| 1 | 50 mM DTT | 1.84 × 10¹⁴ |
| 2 | 100 mM DTT | 2.19 × 10¹⁴ |
| 3 | 100 mM THP | 1.33 × 10¹⁴ |
| 4 | 200 mM THP | 1.63 × 10¹⁴ |

### Example 59

### Selective Capture and Release of Human Breast Cancer Cells (MCF-7) Using Adsorbent Bearing Transferrin

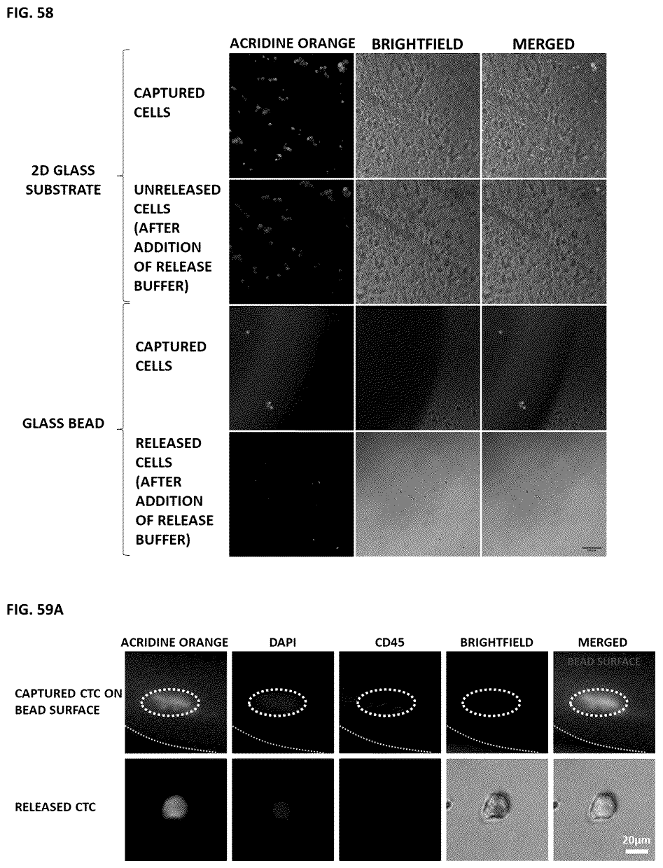

MCF-7 cells (5×10 3) were stained with acridine orange (AO, 40 μM) and 4′,6-diamidino-2-phenylindole (DAPI, 15 μM) and incubated with glass beads of Example 6, and on a glass slide. Cells were incubated for 15 min and 45 min with these beads and on the glass slide respectively. Subsequently, the beads and glass slide were washed with 1×PBS (pH 7.4) three times and incubated with the release buffer (50 mM DTT in 0.1 M sodium bicarbonate buffer, pH 8.0) for the release of the cells. The selective capture and release of the cells was observed in both cases (See FIG. 58).

### Example 60

### Selective Capture, Release and Isolation of Single Circulating Tumor Cells (CTCs) Using Adsorbent Bearing Transferrin

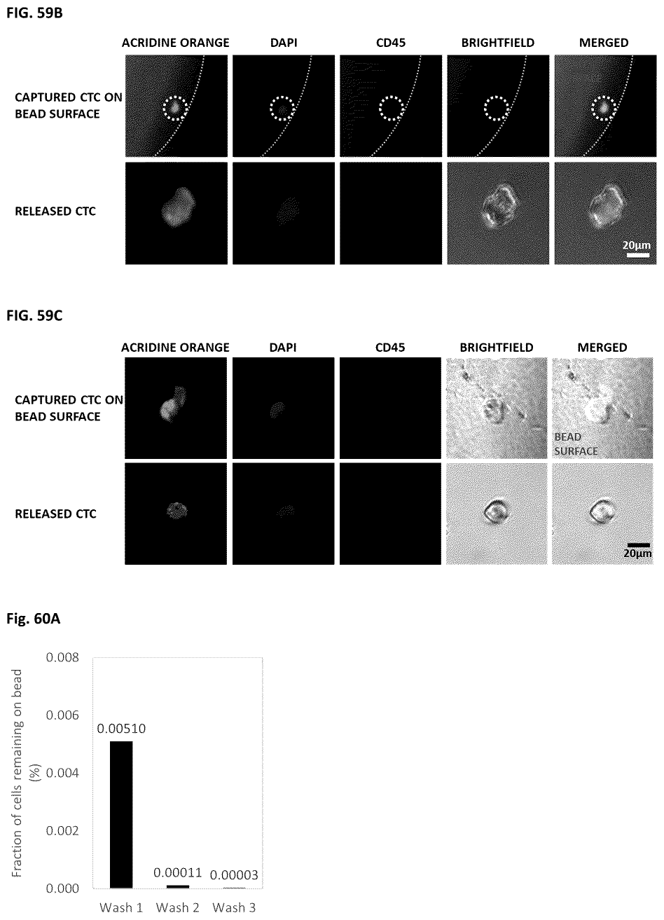

200 μl blood from a cancer patient was incubated at 25° C. for 20 min with the glass beads of example 54 bearing transferrin. Subsequently, the beads were washed three times with 1×PBS (pH 7.4). Captured cells were stained with AO, DAPI and anti-CD45 antibody. These beads were incubated at 37° C. for 20 min with the release buffer as in Example 59 for the release of captured cells (See FIG. 59A).

In another experiment, 3 ml whole blood from a cancer patient was processed for RBC lysis by mixing with 6 ml 1×RBC lysis buffer and incubated for 10 min at 25° C. on a rotary shaker. The mixture was centrifuged at 500×g for 5 min at 25° C. The supernatant was discarded and the pellet was resuspended in 1 ml 1×RBC lysis buffer. The resuspended mixture was incubated at 25° C. for 5 min followed by centrifugation as mentioned above. The pelleted cells were washed twice with 1×PBS (pH 7.4) to remove lysed RBCs, and were then stained with AO, DAPI and anti-CD45 antibody. The cells were incubated with the glass beads of Example 54 bearing transferrin (25° C., 20 min). These glass beads were washed three times with 1×PBS (pH 7.4) and incubated at 37° C. for 20 min with the release buffer as in example 59 for the release of captured cells (See FIGS. 59B and 59C).

AO and DAPI positive, but CD45 negative cells were identified as CTCs and cells positive for prominent CD45 signal along DAPI and AO were identified as leukocytes.

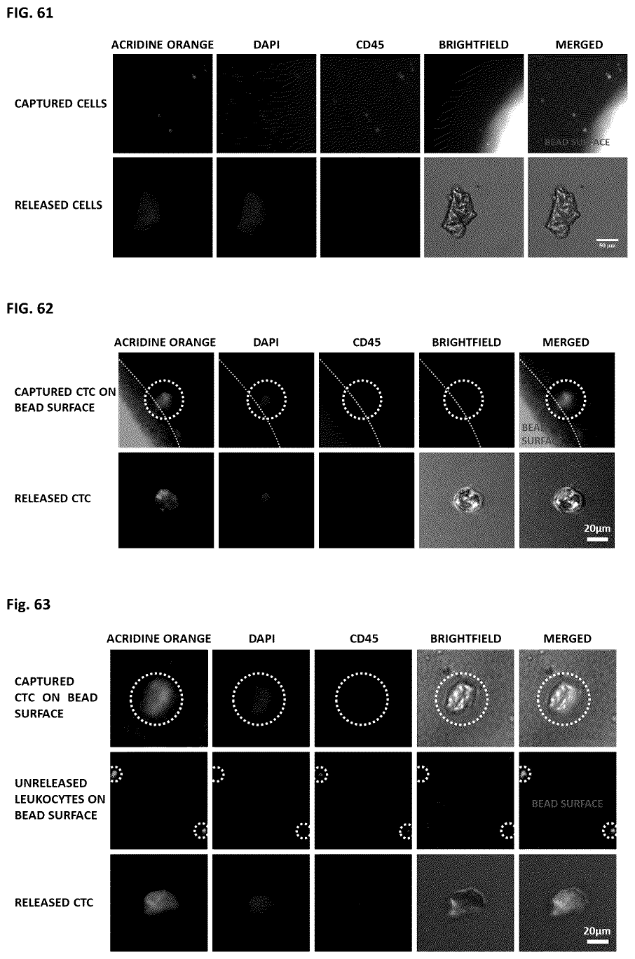

Individual single CTCs from release buffer were collected into a confined volume buffer ranging from 0.2 μL to 200 μL by manual pipetting while visualizing with a fluorescence microscope, and were stored at −80° C. (See FIGS. 61, 62, and 63). Also, individual single CTC isolated from non-small cell lung cancer patient's blood sample (See FIG. 59C).

### Example 61

### Eluting Non-Specifically Bound Cells Using Phosphate Buffer

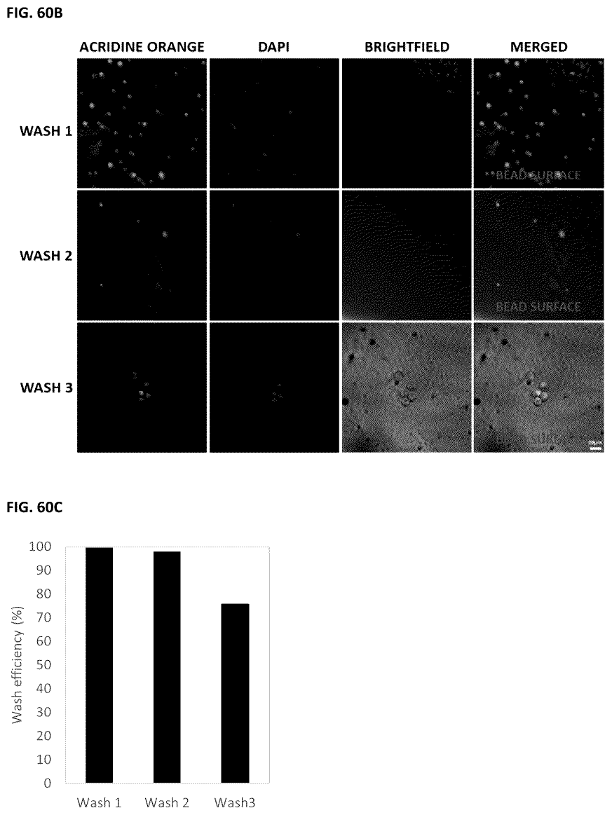

200 μl processed blood from a cancer patient (spiked with MCF-7 cells) was incubated at 25° C. for 15 min. with the transferrin conjugated glass beads of Example 6 The glass beads were then washed three times with 1×PBS (pH 7.4). After each wash, the number of non-specifically attached cells eluted during washing, and those retained on the bead surface were counted separately. FIGS. 60A and 60B show the fraction of cells that remained attached on the bead surface after each wash with respect to the total number of cells attached to the bead initially. FIG. 60C shows the efficiency of removal of non-specifically bound cells through serial washes. 99.99% of the non-specifically attached cells were removed with the serial buffer washes

### Example 62

### Selective Capture and Release of Spiked MCF-7 Cells from the Blood of a Healthy Individual Using Adsorbent Bearing Transferrin

1×10 3 MCF-7 cells were stained with AO and DAPI and added to 200 μl of the whole blood from a healthy individual. Cell capture and release was carried out as in Example 60 (See FIG. 61). MCF-7 cells were captured, released, and stored at −80° C.

### Example 63

### Selective Capture and Release of CTCs Using Adsorbent Bearing Anti-EpCAM Antibody

CTCs from a cancer patient's whole blood were captured and released using glass beads of Example 6 bearing anti-EpCAM antibody. The protocol for capture and release followed was as per Example 60 (See FIG. 62). CTCs were captured, released, and stored at −80° C.

### Example 64

### Capture and Selective Release of CTCs Using Adsorbent Bearing Anti-EpCAM Through Cleavable Linkage and Anti-CD45 Through Non-Cleavable Linkage

CTCs from a cancer patient's processed blood were captured and released using glass beads of example 51 bearing anti-EpCAM (cleavable) and anti-CD45 (non-cleavable) antibodies. The protocol for capture and release followed was according to Example 60. Leukocytes were adsorbed onto the bead's surface. Consequently, the presence of leukocytes in the solution was reduced, allowing for efficient isolation of individual CTCs. The isolated CTCs were stored at −80° C. (See FIG. 63).

### Example 65

### Capture and Selective Release of Leukocytes Using Adsorbent Bearing Terminal Anti-CD45 Antibody Through Cleavable Linkage and Anti-EpCAM Antibody Through Non-Cleavable Linkage

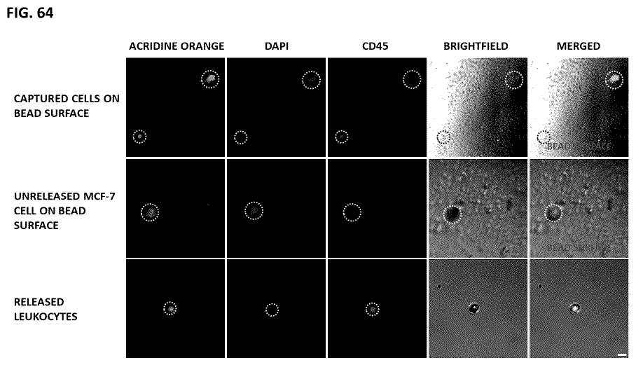

Cancer cells spiked in a blood sample were treated with glass beads of Example 52 bearing anti-CD45 and anti-EpCAM antibodies. The protocol for capture and release followed was according to Example 60. Leukocytes were released while cancer cells remained attached to the beads post incubation with the release buffer (See FIG. 64).

### Example 66

### Selective Capture, Release and Collection of Single CTCs Using Adsorbent Bearing Transferrin Ligand

CTCs from a cancer patient's processed blood were captured and released using glass beads of Example 45 bearing transferrin ligand. The protocol for capture followed was according to Example 60. For CTCs release, the beads were incubated with the release buffer (1 U urease enzyme per 100 μL) at 25° C. for 20 min. The isolated CTCs were stored at −80° C.

### Example 67

### Selective Capture, Release and Collection of Single Circulating Tumor Cells (CTCs) Using Adsorbent Bearing Transferrin Ligand

CTCs from a cancer patient's processed blood were captured and released using glass beads of Example 49 bearing transferrin ligand. The protocol for capture followed was according to Example 60. For CTCs release, the beads were irradiated with visible light (350-550 nm) at 25° C. for 20 min. The isolated CTCs were stored at −80° C.

What is claimed is:

1. An adsorbent composition comprising a functionalized substrate, (1) covalently coupled through a spacer to a ligand that binds non-covalently to a marker cancer cell, and (2) covalently coupled through a spacer to a ligand that binds non-covalently to leukocytes.

2. The adsorbent composition as claimed in claim 1, wherein the functionalized substrate is covalently coupled through at least two spacers to a ligand that binds non-covalently to the marker cancer cell, wherein at least two spacers are linked to each other through a covalent linkage that is cleavable.

3. The adsorbent composition as claimed in claim 1, wherein the functionalized substrate is covalently coupled through a spacer to a ligand that binds non-covalently to the marker cancer cell, wherein the covalent linkage is non-cleavable.

4. The adsorbent composition as claimed in claim 1, wherein the functionalized substrate is covalently coupled through a spacer to a ligand that binds non-covalently to leukocytes through a covalent linkage that is cleavable.

5. The adsorbent composition as claimed in claim 1, wherein the functionalized substrate is covalently coupled through at least two spacers to a ligand that binds non-covalently to leukocytes, wherein the at least two spacers are linked to each other through a covalent linkage that is cleavable.

6. The adsorbent composition as claimed in claim 1, wherein the functionalized substrate is obtained by treating the substrate using a functionalizing agent.

7. The adsorbent composition as claimed in claim 1, wherein the functionalized substrate is obtained by treating the substrate using Piranha solution.

8. The adsorbent composition as claimed in claim 1, wherein the functionalized substrate is selected from functionalized glass and functionalized graphene oxide.

9. The adsorbent composition as claimed in claim 1, wherein the functionalized substrate is selected from functionalized glass beads and functionalized glass slide.

10. The adsorbent composition as claimed in claim 1, wherein the functionalized substrate is hydroxylated glass.

11. The adsorbent composition as claimed in claim 1, wherein the functionalized substrate is selected from functionalized iron oxide and functionalized graphene oxide.

12. The adsorbent composition as claimed in claim 11, wherein the functionalized substrate is obtained by treating iron oxide and graphene oxide with a functionalizing agent selected from (3-aminopropyl) triethoxysilane (APTES), (3-glycidyloxypropyl) trimethoxy silane (GPTMS), (3-glycidyloxypropyl)triethoxysilane (GPTES), (3-mercaptopropyl) trimethoxy silane (MPTMS), (3-mercaptopropyl) triethoxysilane (MPTES), (3-Thiocyanatopropyl) trimethoxysilane (TCPTES), (3-Isocyanatopropyl) triethoxysilane (ICPTES), and combinations thereof.

13. The adsorbent composition as claimed in claim 1, wherein the spacer is selected from 2,2′-dithiodipyridine (DTDP), 5,5′-dithiobis(2-nitrobenzoic acid) (DTNB), N-succinimidyl 3-(2-pyridyldithio)propionate) (SPDP), Poly(ethylene glycol) 2-mercaptoethyl ether acetic acid (SH-PEG-COOH), SPDP-PEG₃₆-NHS ester, 3-mercaptopropionic acid (MPA), 6-maleimidohexanoic acid glutathione, cysteine, and combinations thereof.

14. The adsorbent composition as claimed in claim 1, wherein the spacer is selected from (3-aminopropyl)triethoxysilane (APTES), (3-glycidyloxypropyl)trimethoxysilane (GPTMS), (3-glycidyloxypropyl)triethoxysilane (GPTES), (3-mercaptopropyl)trimethoxy silane (MPTMS), (3-mercaptopropyl)triethoxysilane (MPTES), (3-Thiocyanatopropyl)trimethoxysilane (TCPTES), (3-Isocyanatopropyl)triethoxysilane (ICPTES), and combinations thereof.

15. The adsorbent composition as claimed in claim 1, wherein the marker cancer cell is selected from a circulating tumour cell and a cancer stem cell.

16. The adsorbent composition as claimed in claim 1, wherein the ligand that binds non-covalently to a marker cancer cell is selected from anti-epithelial cell adhesion molecule antibody (anti-EpCAM), and transferrin.

17. The adsorbent composition as claimed in claim 1, wherein the ligand that binds noncovalently to leukocytes is anti-CD45 antibody.

18. The adsorbent composition as claimed in claim 2, wherein the covalent linkage that is cleavable is cleaved in the presence of a stimulus selected from a reducing agent, an enzyme, and irradiation.

19. The adsorbent composition as claimed in claim 18, wherein the reducing agent is selected from dithiothreitol, tris (hydroxypropyl) phosphine, 2-mercaptoethanol, and combinations thereof.

20. The adsorbent composition as claimed in claim 19, wherein the reducing agent is in a buffer selected from sodium phosphate buffer, sodium bicarbonate buffer, tris buffered saline (TBS), and combinations thereof.

21. The adsorbent composition as claimed in claim 2, wherein the covalent linkage that is cleavable is cleaved in the presence of an enzyme selected from urease and cathepsin-B.

22. The adsorbent composition as claimed in claim 2, wherein the covalent linkage that is cleavable is cleaved in the presence of light irradiation in the frequency range of from 350 nm to 550 nm.

23. The adsorbent composition as claimed in claim 2, wherein the covalent linkage that is cleavable is selected from a disulfide linkage, a urea linkage, and a tetrazole ring.

24. The adsorbent composition as claimed in claim 3, wherein the covalent linkage that is non-cleavable is selected from an amino-alcohol linkage, and a thiol-maleimide linkage.

25. The adsorbent composition as claimed in claim 1, for the isolation of a single marker cell for cancer, wherein the said isolated single isolated single marker cell for cancer is free from any fixing agent.

26. A method of isolating a single marker cell for cancer from the blood of a cancer patient comprising the steps of: (1) providing blood from a cancer patient; (2) lysing RBCs by mixing the blood with RBC lysis buffer; (3) incubating and centrifuging the mixture to form a supernatant and a pellet of cells; (4) discarding the supernatant and resuspending the pellet of cells in RBC lysis buffer; (5) incubating the resuspended mixture and centrifuging form a pellet of cells; (6) washing the pelleted cells; (7) staining with a staining agent; (8) incubating the cells with the adsorbent composition of claim 1; (9) washing the adsorbent; (10) incubating in the presence of a stimulus that releases the marker cell, and (11) identifying and isolating the released marker cell.

27. The method of claim 26 wherein the stimulus that releases the marker cell is selected from a reducing agent, an enzyme, and irradiation.

28. The method of claim 27, wherein the reducing agent is selected from dithiothreitol tris (hydroxypropyl) phosphine, and 2-mercaptoethanol.

29. The method of claim 28, wherein the reducing agent is in a buffer selected from sodium phosphate buffer, sodium bicarbonate buffer, tris buffered saline (TBS), and combinations thereof.

30. The method of claim 27, wherein the stimulus that releases the marker cell is an enzyme selected from urease and cathepsin-B.

31. The method of claim 27, wherein the stimulus that releases the marker cell is irradiation in the range selected of from 350 nm to 550 nm.

32. The method for the isolation of a single marker cell for cancer as claimed in claim 26, wherein the said isolated single marker cell for cancer is free from any fixing agent.
